(19) United States
(12) Patent Application Publication
(10) Pub. No.: US 2018/0236255 A1
(43) Pub. Date: Aug. 23, 2018

(54) NEUROSTIMULATION TREATMENT

(71) Applicant: The Board of Trustees of the Leland Standford Junior University, Palo Alto, CA (US)

(72) Inventor: Amit Etkin, Palo Alto, CA (US)

(21) Appl. No.: 15/903,617

(22) Filed: Feb. 23, 2018

Related U.S. Application Data
(60) Provisional application No. 62/462,811, filed on Feb. 23, 2017.

(51) Int. Cl. A61N 2/00; A61N 2/02

(52) U.S. Cl. CPC A61N 2/006; A61B 5/0484; A61N 2/02

(57) ABSTRACT

The disclosures provided herewith relate to systems and methods for mapping personalized brain circuits (or brain network) and/or any abnormalities therein for an individual subject and remediating a treatment protocol based on the personalized map.

### CROSS-REFERENCES TO RELATED APPLICATIONS

This application claims priority to U.S. Provisional Application No. 62/462,811, filed Feb. 23, 2017, which is hereby incorporated by reference in its entirety and for all purposes.

### FIELD OF THE INVENTION

The disclosures provided herewith relate, inter alia, to diagnosis and treatment of mental disorders.

### BACKGROUND OF THE INVENTION

Mental disorders, conditions or illnesses are the largest source of healthcare utilization costs in the U.S., and the costliest of non-communicable diseases worldwide—estimated to result in $6 Trillion in annual societal burden by 2030. Provided herein are solutions to these and other problems in the art.

### BRIEF SUMMARY OF THE INVENTION

In a first aspect, there is provided a method of treating a brain dysfunction in a subject in need thereof. The method includes administering a first plurality of non-invasive stimulations to a first plurality of different brain regions of the subject, measuring a first plurality of responses to the first plurality of non-invasive stimulations, determining a dysfunctional brain network circuit in the subject based on the measuring of the first plurality of responses, and administering a second plurality of non-invasive stimulations, based on the determining of the dysfunctional brain network circuit, to a second plurality of different brain regions of the subject, thereby treating the brain dysfunction in the subject.

In a second aspect, there is provided a method of treating a brain dysfunction in a subject in need thereof. The method includes administering a first plurality of non-invasive stimulations to a first plurality of different brain regions of the subject, measuring a first plurality of responses to the first plurality of non-invasive stimulations, thereby determining a brain region response for the subject, and administering a second plurality of non-invasive stimulations, based on the brain region response, to a second plurality of different brain regions of the subject thereby treating the brain dysfunction. The second plurality of non-invasive stimulations is a subset of the first plurality of non-invasive stimulations and the second plurality of different brain regions is a subset of the first plurality of different brain regions.

In an aspect, there is provided a method of detecting a brain abnormality in a subject that has not been diagnosed with a clinical brain dysfunction or disorder. The method includes administering a non-invasive stimulation to a plurality of different brain regions of the subject, wherein the non-invasive stimulation to each of the plurality of different brain regions is optionally different, measuring a response to said non-invasive stimulation in each of said plurality of different brain regions thereby obtaining a measured response in each of said plurality of different brain regions; and identifying the measured response as being an abnormal response by at least applying, to the measured response, a machine learning model, thereby detecting a brain abnormality.

In an aspect, there is provided a method of detecting a brain abnormality in a subject that has not been diagnosed with a clinical brain dysfunction or disorder. The method includes administering a non-invasive stimulation to a plurality of different brain regions of the subject, wherein the non-invasive stimulation to each of the plurality of different brain regions is optionally different, measuring a response to said non-invasive stimulation in each of said plurality of different brain regions thereby obtaining a measured response in each of said plurality of different brain regions; and comparing the measured response to a control response thereby detecting a brain abnormality in the subject.

### BRIEF DESCRIPTION OF THE DRAWINGS

FIGS. 1A-1D show diagrams illustrating casual circuit interrogation and modulation work. FIG. 1A) Single pulse transcranial magnetic stimulation/Functional magnetic resonance imaging (spTMS/fMRI): posterior dorsal lateral prefrontal cortex (pDLPFC) causally controls network connectivity. FIG. 1B) spTMS/fMRI: TMS to the ventrolateral prefrontal cortex (vlPFC) normally dampens amygdala activity, but fails to do so in Post Traumatic Stress Disorder (PTSD). FIG. 1C) spTMS/EEG (electroencephalogram): excessive network rebound to stimulation in network/memory-impaired PTSD. FIG. 1D) spTMS/EEG: prediction and tracking of circuit change with repetitive TMS (rTMS) treatment.

FIG. 2 shows a diagram illustrating an overview of the procedures described in Example 3.

### DETAILED DESCRIPTION OF THE INVENTION

In some aspects, the disclosures provided herewith relate, inter alia, to systems and methods for mapping (or determining) personalized brain circuits (or brain network) and/or any abnormalities therein for an individual subject and using the personalized map to determine and/or optimize a treatment protocol for a mental disorder deemed to affect the individual subject.

The way in which the field has generally defined psychiatric diagnoses (i.e. based substantially on symptom clusters) and identified treatments (i.e. capitalizing on serendipity), has failed to substantially mitigate the disabling burden of these diseases (disorders, conditions or illnesses), which often appear early in life and persist. Not surprisingly, individual psychiatric diagnoses are often clinically and biologically heterogeneous, with as much or greater variability within a diagnosis as between diagnoses. The number of mechanistically distinct psychiatric drug targets has also not grown in decades, and typically only half of patients respond well in clinical trials. Neuroimaging, as a tool in human neuroscience, however, has been used largely for comparing these arbitrarily defined diagnoses against healthy individuals, not for robustly characterizing individual patients in objective biological terms. Imaging is also a purely observational method, and thus cannot by itself provide the causal understanding of circuitry that is necessary for transitioning from a descriptive to a circuit-based mechanistic understanding of mental illness that can directly guide novel interventions.

The conventional methods for neurostimulation treatment in disorders of the central nervous system are “one-size-fits-all” (one intervention for a particular diagnosis), and not based on any information regarding the individual's brain. The current diagnoses in psychiatry, neurology and chronic pain are therefore often poorly defined, rely on self-reported symptoms, and do not align well with brain-based pathophysiology. Hence, current neurostimulation interventions do not work for many patients due to being under-specified relative to their particular brain abnormalities (or not using the correct method of remediating these abnormalities).

In order to overcome the foregoing issues and meet the needs in the field such as providing an effective and optimized treatment protocol for an individual patient, the inventors conceived a new diagnostic and treatment development framework that transcends the arbitrariness of traditional diagnoses, the limitations of group-level imaging analyses and current trial-and-error approaches to treatment planning. Rather, this platform, also referred to as the “Circuits-First” platform herein, relies on understanding causality in the brain circuits of individual patients as a means for personalized diagnosis and treatment using individually-tailored circuit-targeting interventions. The term “casualty” may refer to one or multiple signals in the brain that arise as a consequence of stimulation to a region (i.e. a causal consequence of that stimulation). Therefore, the disclosures herewith provide systems and methods for carrying out individualized causal circuitry assessment, and development and/or optimization of treatment, where circuit function is therapeutically modified through plasticity-inducing neurostimulation and a direct linkage between circuits and clinical outcome is established. The term “plasticity” may refer to changes in brain function that may result from repetitive stimulation to one or multiple regions. In embodiments, these changes in brain function may outlast the period of repetitive stimulation and can be seen in the brain response evoked by stimulation or in alteration of the brain response to a stimulus or while at rest. The disclosures herewith are relied, at least in part, on the understanding of causality in human brain circuits (e.g. how manipulating activity in one brain region affects activity in another) by combining focal non-invasive neurostimulation with concurrent neuroimaging as well as the capability of defining circuit aberrations at the individual patient level. Consequently, the disclosures herewith can establish a platform for rapid translation to other psychiatric disorders, and applicable to various neurological disorders (e.g. stroke, Parkinson's) where circuit perturbations are deemed involved or relevant.

The signaling in a biological neural network is based on a highly coordinated system of electric charges, neurotransmitters and action potentials. The ability to reliably and non-invasively incite and monitor neuronal activity changes from outside the head with the purpose of modulating activity in specific neural networks remains a roadblock to enable advances in the detection, monitoring, and treatment of psychiatric, neurological and related conditions. A biological neural network can be considered as a complex electrical circuit made of many neurons connected through synapses formed between axons and dendrites. Both types of synapses, known as chemical and electrical synapses, respectively, transfer information between adjacent axons and dendrites directly or indirectly through electric field energy. Consequently, the biological neural network is sensitive to external electric fields.

Non-invasive brain stimulation locally alters brain electrical signaling. These local alterations in signaling can result in broader alterations to neuronal signaling throughout the brain. These circuit-wide effects of non-invasive brain stimulation reflect the brain effects of stimulation as well as the network rebound response to a burst of activity entering the system. This set of events is referred to herein as a non-invasive brain stimulation evoked response (e.g., a TMS evoked response) or simply response.

### Definitions

Unless specifically stated or obvious from context, as used herein, the term “or” is understood to be inclusive.

Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Ranges provided herein are understood to be shorthand for all of the values within the range.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The terms “brain dysfunction”, “brain abnormality” or “brain disorder” may refer to any type or degree of impairment in brain function that can affect, for example, perception, behavior, motion or mobility, and/or other abilities, such as learning and memory, and can be characterized by one or more of various symptoms, e.g. dyslexia, difficulty in writing, hyperactivity, mental retardation, affecting consciousness, making people drowsy, difficulty to arouse (e.g. causing stupor or coma), and more. A brain dysfunction, abnormality, or disorder may result from a variety of factors, including, but not limited to, genetics, the environment, or a combination of the two. A brain dysfunction, abnormality, or disorder may be diagnosed clinically (e.g., a clinical brain dysfunction or disorder). In embodiments, a brain abnormality, dysfunction, or disorder may be detected (e.g., identified) by assaying neural activity in a brain region or regions and/or brain circuits. In embodiments, the term “brain” as used herein refers to a human brain.

The term “brain region(s)” is used according to its plain and ordinary meaning and refers to a brain anatomical region following standard neuroanatomy hierarchies (e.g. a functional, connective or developmental region). Exemplary brain regions include, but are not limited to, brainstem, Medulla oblongata, Medullary pyramids, Olivary body, Inferior olivary nucleus, Rostral ventrolateral medulla, Respiratory center, Dorsal respiratory group, Ventral respiratory group, Pre-Bötzinger complex, Botzinger complex, Paramedian reticular nucleus, Cuneate nucleus, Gracile nucleus, Intercalated nucleus, Area postrema, Medullary cranial nerve nuclei, Inferior salivatory nucleus, Nucleus ambiguus, Dorsal nucleus of vagus nerve, Hypoglossal nucleus, Solitary nucleus, Pons, Pontine nuclei, Pontine cranial nerve nuclei, chief or pontine nucleus of the trigeminal nerve sensory nucleus (V), Motor nucleus for the trigeminal nerve (V), Abducens nucleus (VI), Facial nerve nucleus (VII), vestibulocochlear nuclei (vestibular nuclei and cochlear nuclei) (VIII), Superior salivatory nucleus, Pontine tegmentum, Respiratory centers, Pneumotaxic center, Apneustic center, Pontine micturition center (Barrington's nucleus), Locus coeruleus, Pedunculopontine nucleus, Laterodorsal tegmental nucleus, Tegmental pontine reticular nucleus, Superior olivary complex, Paramedian pontine reticular formation, Cerebellar peduncles, Superior cerebellar peduncle, Middle cerebellar peduncle, Inferior cerebellar peduncle, Cerebellum, Cerebellar vermis, Cerebellar hemispheres, Anterior lobe, Posterior lobe, Flocculonodular lobe, Cerebellar nuclei, Fastigial nucleus, Interposed nucleus, Globose nucleus, Emboliform nucleus, Dentate nucleus, Tectum, Corpora quadrigemina, inferior colliculi, superior colliculi, Pretectum, Tegmentum, Periaqueductal gray, Parabrachial area, Medial parabrachial nucleus, Lateral parabrachial nucleus, Subparabrachial nucleus (Kölliker-Fuse nucleus), Rostral interstitial nucleus of medial longitudinal fasciculus, Midbrain reticular formation, Dorsal raphe nucleus, Red nucleus, Ventral tegmental area, Substantia nigra, Pars compacta, Pars reticulata, Interpeduncular nucleus, Cerebral peduncle, Crus cerebri, Mesencephalic cranial nerve nuclei, Oculomotor nucleus (III), Trochlear nucleus (IV), Mesencephalic duct (cerebral aqueduct, aqueduct of Sylvius), Pineal body, Habenular nucleim Stria medullares, Taenia thalami, Subcommissural organ, Thalamus, Anterior nuclear group, Anteroventral nucleus (aka ventral anterior nucleus), Anterodorsal nucleus, Anteromedial nucleus, Medial nuclear group, Medial dorsal nucleus, Midline nuclear group, Paratenial nucleus, Reuniens nucleus, Rhomboidal nucleus, Intralaminar nuclear group, Centromedial nucleus, Parafascicular nucleus, Paracentral nucleus, Central lateral nucleus, Central medial nucleus, Lateral nuclear group, Lateral dorsal nucleus, Lateral posterior nucleus, Pulvinar, Ventral nuclear group, Ventral anterior nucleus, Ventral lateral nucleus, Ventral posterior nucleus, Ventral posterior lateral nucleus, Ventral posterior medial nucleus, Metathalamus, Medial geniculate body, Lateral geniculate body, Thalamic reticular nucleus, Hypothalamus, limbic system, HPA axis, preoptic area, Medial preoptic nucleus, Suprachiasmatic nucleus, Paraventricular nucleus, Supraoptic nucleusm Anterior hypothalamic nucleus, Lateral preoptic nucleus, median preoptic nucleus, periventricular preoptic nucleus, Tuberal, Dorsomedial hypothalamic nucleus, Ventromedial nucleus, Arcuate nucleus, Lateral area, Tuberal part of Lateral nucleus, Lateral tuberal nuclei, Mammillary nuclei, Posterior nucleus, Lateral area, Optic chiasm, Subfornical organ, Periventricular nucleus, Pituitary stalk, Tuber cinereum, Tuberal nucleus, Tuberomammillary nucleus, Tuberal region, Mammillary bodies, Mammillary nucleus, Subthalamus, Subthalamic nucleus, Zona incerta, Pituitary gland, neurohypophysis, Pars intermedia, adenohypophysis, cerebral hemispheres, Corona radiata, Internal capsule, External capsule, Extreme capsule, Arcuate fasciculus, Uncinate fasciculus, Perforant Path, Hippocampus, Dentate gyms, Cornu ammonis, Cornu ammonis area 1, Cornu ammonis area 2, Cornu ammonis area 3, Cornu ammonis area 4, Amygdala, Central nucleus, Medial nucleus (accessory olfactory system), Cortical and basomedial nuclei, Lateral and basolateral nuclei, extended amygdala, Stria terminalis, Bed nucleus of the stria terminalis, Claustrum, Basal ganglia, Striatum, Dorsal striatum (aka neostriatum), Putamen, Caudate nucleus, Ventral striatum, Striatum, Nucleus accumbens, Olfactory tubercle, Globus pallidus, Subthalamic nucleus, Basal forebrain, Anterior perforated substance, Substantia innominata, Nucleus basalis, Diagonal band of Broca, Septal nuclei, Medial septal nuclei, Lamina terminalis, Vascular organ of lamina terminalis, Olfactory bulb, Piriform cortex, Anterior olfactory nucleus, Olfactory tract, Anterior commissure, Uncus, Cerebral cortex, Frontal lobe, Frontal cortex, Primary motor cortex, Supplementary motor cortex, Premotor cortex, Prefrontal cortex, frontopolar cortex, Orbitofrontal cortex, Dorsolateral prefrontal cortex, dorsomedial prefrontal cortex, ventrolateral prefrontal cortex, Superior frontal gyms, Middle frontal gyms, Inferior frontal gyms, Brodmann areas: 4, 6, 8, 9, 10, 11, 12, 24, 25, 32, 33, 44, 45, 46, 47, Parietal lobe, Parietal cortex, Primary somatosensory cortex (S1), Secondary somatosensory cortex (S2), Posterior parietal cortex, postcentral gyms, precuneus, Brodmann areas 1, 2, 3 (Primary somesthetic area); 5, 7, 23, 26, 29, 31, 39, 40, Occipital lobe, Primary visual cortex (V1), V2, V3, V4, V5/MT, Lateral occipital gyms, Cuneus, Brodmann areas 17 (V1, primary visual cortex); 18, 19, temporal lobe, Primary auditory cortex (A1), secondary auditory cortex (A2), Inferior temporal cortex, Posterior inferior temporal cortex, Superior temporal gyms, Middle temporal gyms, Inferior temporal gyms, Entorhinal Cortex, Perirhinal Cortex, Parahippocampal gyms, Fusiform gyms, Brodmann areas: 9, 20, 21, 22, 27, 34, 35, 36, 37, 38, 41, 42, Medial superior temporal area (MST), insular cortex, cingulate cortex, Anterior cingulate, Posterior cingulate, dorsal cingulate, Retrosplenial cortex, Indusium griseum, Subgenual area 25, Brodmann areas 23, 24; 26, 29, 30 (retrosplenial areas); 31, 32, cranial nerves (Olfactory (I), Optic (II), Oculomotor (III), Trochlear (IV), Trigeminal (V), Abducens (VI), Facial (VII), Vestibulocochlear (VIII), Glossopharyngeal (IX), Vagus (X), Accessory (XI), Hypoglossal (XII)), and neural pathways Superior longitudinal fasciculus, Arcuate fasciculus, Thalamocortical radiations, Cerebral peduncle, Corpus callosum, Posterior commissure, Pyramidal or corticospinal tract, Medial longitudinal fasciculus, dopamine system, Mesocortical pathway, Mesolimbic pathway, Nigrostriatal pathway, Tuberoinfundibular pathway, serotonin system, Norepinephrine Pathways, Posterior column-medial lemniscus pathway, Spinothalamic tract, Lateral spinothalamic tract, Anterior spinothalamic tract. Brain regions and specific parts of brain regions may be referred to according to their rostral/caudal, dorsal/ventral, medial/lateral, and/or anterior/posterior positions within the brain region with respect to the skull.

The term “brain network”, “brain circuit”, “brain (or neural) connection” or “brain region connectivity” refers to a plurality of brain regions having activity correlated with each other.

The term “dysfunctional brain network circuit (or connection)” or “dysfunctional brain circuit (network or connection)” may refer to a brain circuit, network or connection that has any detectable type or degree of impairment in brain function. The dysfunctional circuit, network or connection may or may not be accompanied by notable or detectable symptoms. In embodiments, an impairment in brain function includes an impairment in one or more of the following variables: a timing of the response, a magnitude in response, a phase or frequency of the response, a duration of the response, and/or a presence or an absence of the response, to one or more non-invasive stimulations to one or more brain regions of a subject. In embodiments, a variable is impaired when it deviates (e.g., positively or negatively) from a variable measured in a control subject (e.g., healthy control) by at least 5%, 10%, 15%, 20%, 30%, 40%, 50%, 60%, 70%, 80%, 90%, 100% or more. The variables as described herein may be elicited by different stimulation techniques (e.g., TMS, fUS, tDCS, tACS) and stimulation paradigms (e.g., repetitive stimulation, burst stimulation, single pulse stimulation, paired pulse stimulation) administered to one or more brain regions, and measured in one or more of the same or different brain regions using a variety of techniques, such as, for example, EEG, MEG, fMRI, or NIRS. It will be obvious to a person of ordinary skill in the art that measurements of the variables will necessary differ depending on factors including, but not limited to, stimulation technique, stimulation paradigm, the brain region or regions stimulated, response recording technique, and the brain region or regions where response is recorded. Thus, measurements of variables made in an effort to determine impairment or abnormality of a brain function in a subject must be compared against identical stimulation and recording procedures made in control subjects.

The term “transcranial magnetic stimulation” or “TMS” as used herein refers to a non-invasive brain stimulation method which employs a magnetic field generator applied near the head to locally stimulate an electrical current within the brain. In embodiments, TMS includes repetitive transcranial magnetic stimulation or rTMS. Treatment with rTMS is comprised of multiple sessions (either daily across days or multiple times per day and across days) wherein TMS is delivered repetitively in a pattern that is intended to induce plasticity (defined as a change in brain activity). This plasticity could increase or decrease the activity of the brain region that is targeted. In embodiments, the rTMS is a “high frequency” protocol, involving stimulation at >5 Hz (e.g., 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 250, 300 Hz or higher). In embodiments, the rTMS is a “low frequency” protocol, involving stimulation at <1 Hz (e.g., 0.9, 0.8, 0.7, 0.6, 0.5, 0.4, 0.3, 0.2, 0.1, 0.05 Hz or lower). In embodiments, the rTMS is a “theta burst” protocol, involving stimulation with either a continuous or intermittent theta burst pattern. In embodiments, the rTMS provides a protocol involving stimulations having more than one frequency. In embodiments, rTMS is administered to treat a brain abnormality or dysfunctional brain network circuit.

In embodiments, TMS includes single pulse TMS (spTMS). Single pulse TMS refers to administration of a single pulse with a duration typically on the order of 250 microseconds. In embodiments, the single pulse duration is about 250 microseconds. In embodiments, the single pulse duration is 250 microseconds. In embodiments, TMS includes paired pulse TMS. Paired pulse TMS refers to two pulses delivered with a timing between the single pulses that may be short (e.g. 1, 2, 3 up to 50 ms) or long (e.g. 50 ms or longer, up to 500 ms) in duration. The period in between pulses within the paired pulse sequence can allow interrogation of different neurophysiological processes.

The term “transcranial alternating current stimulation (tACS)” as used herein refers to a non-invasive form of brain stimulation, where a small, pulsed, alternating current is delivered to specific areas of the brain via electrodes positioned on and about the head to influence (e.g., stimulate) neural activity in the targeted brain tissue.

The term “transcranial direct current stimulation (tDCS)” as used herein refers to a non-invasive form of brain stimulation, where low levels of constant current are delivered to specific areas of the brain via electrodes positioned on and about the head to influence (e.g., stimulate) neural activity in the targeted brain tissue.

The term “focused ultrasound stimulation (fUS)”, also referred to as transcranial focused ultrasound stimulation, as used herein refers to a non-invasive form of brain stimulation involving the delivery of acoustic pressure waves to specific, highly localize, and potentially deep areas of the brain, to influence (e.g., stimulate) neural activity in the targeted brain tissue. fUS may be used to target regions of neural tissue on the order of a few millimeters in size.

Non-invasive stimulation of a brain region, multiple brain regions, or a brain circuit (e.g., portion of a brain circuit, all brains regions of a brain circuit) may result in a response (e.g., neural activity response) in the non-invasively stimulated brain region, multiple brain regions, or brain circuit, or a brain region, brain regions, or a brain circuit (e.g., portion of a brain circuit, all brain regions of bran circuit) which were not subject to non-invasive stimulation. The response (e.g., neural activity response) may be measured using a variety of suitable techniques for measuring neural activity known in the art (e.g., EEG, MEG, fMRI, NIRS, fNIRS).

The response (e.g., neural activity response) resulting from the non-invasive stimulation may be an increase in the neural activity relative to a baseline level of neural activity taken prior to administering the non-invasive stimulation. In embodiments, the response may be a decrease in neural activity relative to a baseline level of neural activity taken prior to administering the non-invasive stimulation. In embodiments, the response may be no change in neural activity relative to a baseline level of neural activity taken prior to administering the non-invasive stimulation. It is contemplated that any suitable method of measuring a response (e.g., neural activity) resulting from the non-invasive stimulation may be used (e.g., EEG, MEG, fMRI, NIRS, fNIRS).

In embodiments, a magnitude of a response to non-invasive stimulation is measured. In embodiments, a magnitude of a non-invasive brain stimulation response is measured at 25-50 msecs, 100-150 msecs, or 180 and 200 msecs following non-invasive brain stimulation. The response can be measured between 25-50 msecs (p30), 30-70 msecs (p60), 70-120 msecs (n100), 150-250 msecs (p200). Alternatively, the frequency of the response can be measured. In embodiments, the response may have a frequency of, for example, delta (0.5-4 Hz), theta (5-8 Hz), alpha (8-12 Hz), beta (12-30 Hz), or gamma (30-60 Hz). Similarly, the response can be measured by detecting the amplitude (e.g., power) of oscillations at delta (0.5-4 Hz), theta (5-8 Hz), alpha (8-12 Hz), beta (12-30 Hz), or gamma (30-60 Hz) frequencies. In embodiments, the response frequency, amplitude (e.g., power), and phase can be measured. In embodiments, a duration of the response is measured. In embodiments, a presence or absence of a response is measured. The response may be an average of two or more responses measured. In embodiments, the response may be the median response of two or more responses measured.

In embodiments, a response is an electrical potential or magnetic field recorded from the nervous system, e.g. brain, of a human or other animal, following presentation of a stimulus (e.g., non-invasive stimulation (e.g., TMS, focused ultrasound stimulation, transcranial direct current stimulation, transcranial alternating current stimulation)) that is distinct from spontaneous potentials or fields as detected by electroencephalography (EEG), electromyography (EMG), magnetoencephalography (MEG), or other electrophysiological or neurophysiological recording methods. Such potentials and fields are useful for monitoring brain function in health and disease, and, as described herein, may be used for diagnostic purposes. The recorded electrical potential or magnetic field is often presented with an amplitude, phase and/or frequency, including the amplitude or power of the response frequency, which generally indicates an intensity and/or pattern of the response.

As used herein, the term “electroencephalography (EEG)” refers to a non-invasive neurophysiological technique that uses an electronic monitoring device to measure and record electrical activity in the brain.

As used herein, the term “magnetoencephalography (MEG)” refers to a non-invasive neurophysiological technique that measures the magnetic fields generated by neuronal activity of the brain. The spatial distributions of the magnetic fields are analyzed to localize the sources of the activity within the brain.

Alternatively, in embodiments, a response is a blood flow (e.g., oxygenation level) or blood hemoglobin concentration change as recorded from the nervous system, e.g. brain, of a human or other animal, following presentation of a stimulus (e.g., non-invasive stimulation (e.g., TMS, focused ultrasound stimulation, transcranial direct current stimulation, transcranial alternating current stimulation)), that is distinct from spontaneous blood flow (e.g., oxygenation level) or hemoglobin concentration changes as detected by functional magnetic resonance imaging (fMRI), near-infrared spectroscopy (NIRS, fNIRS), or other suitable methods for detecting changes in blood flow (e.g., oxygenation level) or hemoglobin concentrations associated with changes in neural activity. Such changes are useful for diagnosis and monitoring of brain function in health and disease.

As used herein, the terms “functional magnetic resonance imaging (fMRI)” or “functional MRI (fMRI)” refer to a functional neuroimaging procedure using MRI technology that measures brain activity by detecting changes associated with blood flow.

As used herein, the term “near-infrared spectroscopy (NIRS)” refers to a spectroscopic method that uses the near-infrared region of the electromagnetic spectrum (from about 700 nm to 2500 nm). For example, NIRS (e.g., functional NIRS (fNIRS)) can be used for non-invasive assessment of brain function through the intact skull in human subjects by detecting changes in blood hemoglobin concentrations associated with neural activity.

It should be appreciated that the response as measured in a subject may be compared to a response previously measured in the same subject or to a response measured in a control subject (e.g., healthy control). For example, the response measured in a subject may be compared to a response measured prior to treatment of the subject. In this way, the measured response can serve as an indicator of treatment outcome. Alternatively, the response measured in the subject may be compared to a control subject (e.g., healthy control) for purposes, for example, of diagnosing a brain dysfunction or abnormality.

It is also contemplated that responses, as measured according to embodiments herein, may be classified (e.g., identified) as, for example, a normal response (e.g., a response similar to a control subject (e.g., healthy control)) or an abnormal response (e.g., a response dissimilar to a control subject or healthy control). This type of classification (e.g., identification) may be useful in, for example, detecting a brain abnormality, diagnosing a brain abnormality, determining a course of treatment, and/or determining treatment outcome. Classification may be carried out by, for example, visual inspect and quantification performed by a human operator. Alternatively, classification may be accomplished via human operator-independent means. For example, classification may be accomplished through a computer running a machine learning model (e.g., algorithm) capable of classifying (e.g., identifying) a response as a normal or abnormal response. The machine learning model may be any suitable machine learning model or algorithm known in the art. In embodiments, the model may be trained, for example using training data, to classify a response as abnormal or normal. Training of the algorithm may be accomplished through supervised or unsupervised training methods.

A “subject” as used herein refers to an organism. In certain embodiments, the organism is an animal. In certain embodiments, the subject is a living organism. In certain embodiments, the subject is a cadaver organism. In certain embodiments, the subject is a mammal, including, but not limited to, a human or non-human mammal. In certain embodiments, the subject is a domesticated mammal or a primate including a non-human primate. Examples of subjects include humans, monkeys, dogs, cats, mice, rats, cows, horses, goats, and sheep. A human subject may also be referred to as a patient. In embodiments, the subject suffers from a neurological disorder. In embodiments, the subject suffers from a psychiatric disorder. In embodiments, the subject suffers from posttraumatic stress disorder (PTSD). In embodiments, the subject suffers from depression. In embodiments, the subject suffers from chronic pain. In embodiments, the subject has a brain abnormality. In embodiments, the subject does not have a brain abnormality. In embodiments, the subject has been diagnosed with a brain dysfunction or disorder (e.g., clinical brain dysfunction or disorder). In embodiments, the subject has not been diagnosed with a brain dysfunction or disorder (e.g., clinical brain dysfunction or disorder). In embodiments, the subject is suspected of suffering from a brain dysfunction or disorder. In embodiments, the subject is suspected of suffering from a brain abnormality. In embodiments, the subject is susceptible to suffering from a brain abnormality. In embodiments, the subject is susceptible to suffering from a brain dysfunction or disorder. In embodiments, the subject is susceptible to suffering from a neurological disorder. In embodiments, the subject is susceptible to suffering from a psychiatric disorder. In embodiments, the subject is susceptible to suffering from PTSD. In embodiments, the subject is susceptible to suffering from depression. In embodiments, the subject is susceptible to suffering from chronic pain.

“Control subject”, “healthy control”, “normal non-diseased control subject”, or “standard control”, or the like (e.g., control population), is used in accordance with its plain ordinary meaning and refers herein to a subject not suffering from a disease, condition, syndrome, abnormality, or disorder (e.g., brain dysfunction, brain abnormality, brain disorder). In some instances, the control subject is used as a standard of comparison in evaluating experimental effects. In some instances, the control subject is used as a standard of comparison in the process of diagnosing or prognosing a subject suffering from, suspected of suffering from, not suspected of suffering from, or not clinically diagnosed with a disease, condition, syndrome, abnormality, or disorder (e.g., brain dysfunction, brain abnormality, brain disorder).

A subject “suffering from or suspected of suffering from” a specific disease, disorder, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions associated with cancer is within the ability of those in the art. Subjects suffering from, and suspected of suffering from, a specific disease, disorder, condition, or syndrome are not necessarily two distinct groups. Common risk factors for developing PTSD include, but are not limited to, living through dangerous events and traumas, for example, wars, physical or sexual assault, abuse, accidents, disasters, or other serious events; getting hurt; seeing another person hurt or seeing a dead body; childhood trauma; feeling horror, helplessness, or extreme fear; having little or no social support after a traumatic event; dealing with extra stress after the event, such as loss of a loved one, pain and injury, or loss of a job or home; having a history of mental illness or substance abuse. Common risk factors for developing depression include, but are not limited to, certain personality traits, such as low self-esteem and being too dependent, self-critical or pessimistic; traumatic or stressful events, such as physical or sexual abuse, the death or loss of a loved one, a difficult relationship, or financial problems; blood relatives with a history of depression, bipolar disorder, alcoholism or suicide; history of other mental health disorders, such as anxiety disorder, eating disorders or post-traumatic stress disorder; abuse of alcohol or recreational drugs; serious or chronic illness, including cancer, stroke, chronic pain or heart disease; certain medications, such as some high blood pressure medications or sleeping pills. Common risk factors for developing chronic pain include, but are not limited to, rheumatoid arthritis, osteoarthritis, fibromyalgia, cancer, multiple sclerosis, stomach ulcers, AIDS, and gallbladder disease.

As used herein, “susceptible to” or “prone to” or “predisposed to” a specific disease or condition and the like refers to an individual who based on genetic, environmental, health, and/or other risk factors is more likely to develop a disease, disorder, or condition than the general population. An increase in likelihood of developing a disease may be an increase of about 10%, 20%, 50%, 100%, 150%, 200%, or more.

As used herein, the terms “treat,” treating,” “treatment,” and the like refer to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

The terms “aberrant”, “abnormal”, “impairment”, and the like, as used herein refer to different from normal. When used to describe, for example, responses (e.g., variables as described herein) or brain circuit function, abnormal refers to responses (e.g., variable as described herein) or brain circuit function that is greater or less than a normal non-diseased control subject, the average of normal non-diseased control subjects, wherein an average may be the mean or median of a control population, or a specific amount (e.g., 1.5 standard deviations) outside of the distribution of responses determined in a population of normal non-diseased control subjects. An abnormal response may refer to an amount of activity (e.g., over or under activity (e.g., neural activity), variable as described herein) that results in or is indicative of a disease, wherein returning the abnormal response to a normal response or non-disease-associated response as may be observed in a control subject (e.g. by administering a non-invasive stimulation treatment), results in reduction of the disease or one or more disease symptoms. In embodiments, an abnormal response or impaired response is at least 5%, 10%, 15%, 20%, 30%, 40%, 50%, 60%, 70%, 80%, 90%, 100% or more different (e.g., positively or negatively) from a response measured in a control subject. In embodiments, an abnormal response is a response that falls 1.5 standard deviations or more from the response distribution measured in a control population. In embodiments, an abnormal response or impaired response is at least 5%, 10%, 15%, 20%, 30%, 40%, 50%, 60%, 70%, 80%, 90%, 100% or more different (e.g., positively or negatively) from the average response measured in a control population. In embodiments, an abnormal response or impaired response is at least 5%, 10%, 15%, 20%, 30%, 40%, 50%, 60%, 70%, 80%, 90%, 100% or more different (e.g., positively or negatively) from the median response measured in a control population.

The abnormal response may be identified by comparing one or a combination of variables as described herein in a subject against one or a combination of variables as described herein in a control subject or population. In embodiments, a machine learning model is used to identify an abnormal response.

A “reduction” of a symptom or symptoms (and grammatical equivalents of this phrase) means a decreasing of the severity or frequency of the symptom(s), or elimination of the symptom(s) (e.g. in response to a treatment relative to the absence of the treatment).

The term “therapeutically effective amount,” as used herein, refers to that amount of the therapeutic agent (i.e., non-invasive stimulation) sufficient to ameliorate or reduce symptoms of the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the non-invasive stimulation being employed. The dose administered to a patient, in the context of the present disclosure, should be sufficient to effect a beneficial therapeutic response in the patient over time. The structure of the dose (e.g., rTMS, spTMS, ppTMS) also will be determined by the existence, nature, and extent of any adverse side-effects. Determination of the proper dosage for a particular situation is within the skill of the practitioner. Generally, treatment is initiated with smaller dosages which are less than the optimum dose of the non-invasive stimulation. Thereafter, the dosage is increased by small increments until the optimum effect under circumstances is reached. Dosage amounts (e.g., intensity) and intervals (e.g., treatment sessions) can be adjusted individually to provide levels of the administered non-invasive stimulation effective for the particular clinical indication being treated. This will provide a therapeutic regimen that is commensurate with the severity of the individual's disease state.

As used herein, the term “administering” refers to non-invasive brain stimulation administration. In embodiments, the administering does not include administration of any active agent other than the non-invasive stimulation. In embodiments, the administering includes administration of one or more active agents (e.g., medications) in addition to the non-invasive stimulation.

### Methods of Treatment

In one aspect, there is provided a method of treating a brain dysfunction in a subject in need thereof. The method includes administering a first plurality of non-invasive stimulations to a first plurality of different brain regions of the subject, measuring a first plurality of responses to the first plurality of non-invasive stimulations, determining a dysfunctional brain network circuit in the subject based on the measuring of the first plurality of responses, and administering a second plurality of non-invasive stimulations, based on the determining of the dysfunctional brain network circuit, to a second plurality of different brain regions of the subject, thereby treating the brain dysfunction in the subject.

In another aspect, there is provided a method of treating a brain dysfunction in a subject in need thereof. The method includes administering a first plurality of non-invasive stimulations to a first plurality of different brain regions of the subject, measuring a first plurality of responses to the first plurality of non-invasive stimulations, thereby determining a brain region response for the subject, and administering a second plurality of non-invasive stimulations, based on the brain region response, to a second plurality of different brain regions of the subject thereby treating the brain dysfunction. The second plurality of non-invasive stimulations is a subset of the first plurality of non-invasive stimulations and the second plurality of different brain regions is a subset of the first plurality of different brain regions.

In embodiments, the second plurality of non-invasive stimulations is a subset of the first plurality of non-invasive stimulations and the second plurality of different brain regions is a subset of the first plurality of different brain regions.

In embodiments, the determining a dysfunctional brain network circuit includes comparing the measured responses in the subject to the responses measured in a healthy control. In embodiments, the determining a dysfunctional brain network circuit is performed by a computer executing a machine learning model as described herein. In embodiments, the determining a dysfunctional brain network circuit includes using a machine learning model to classify measured responses in the subject as abnormal or normal.

In embodiments, the first plurality of different brain regions of the subject includes two or more brain regions selected from the group consisting of frontal cortex, temporal cortex, parietal cortex, occipital cortex, hippocampus, amygdala, striatum or brainstem, and subregions thereof.

In embodiments, the first plurality of non-invasive stimulations is transcranial magnetic stimulation (TMS), focused ultrasound stimulation, transcranial direct current or transcranial alternating current stimulation. In embodiments, the first plurality of non-invasive stimulations is TMS. In embodiments, the first plurality of non-invasive stimulations is focused ultrasound stimulation. In embodiments, the first plurality of non-invasive stimulations is transcranial direct current stimulation (tDCS). In embodiments, the first plurality of non-invasive stimulations is transcranial alternating current stimulation (tACS). In embodiments, the first plurality of non-invasive stimulations is one or more (e.g., 1, 2, 3 or 4) selected from the group consisting of transcranial magnetic stimulation (TMS), focused ultrasound stimulation, transcranial direct current and transcranial alternating current stimulation.

In embodiments, the second plurality of non-invasive stimulations is transcranial magnetic stimulation (TMS), focused ultrasound stimulation, transcranial direct current or transcranial alternating current stimulation. In embodiments, the second plurality of non-invasive stimulations is TMS. In embodiments, the second plurality of non-invasive stimulations is focused ultrasound stimulation. In embodiments, the second plurality of non-invasive stimulations is transcranial direct current stimulation (tDCS). In embodiments, the second plurality of non-invasive stimulations is transcranial alternating current stimulation (tACS). In embodiments, the second plurality of non-invasive stimulations is one or more (e.g., 1, 2, 3 or 4) selected from the group consisting of transcranial magnetic stimulation (TMS), focused ultrasound stimulation, transcranial direct current and transcranial alternating current stimulation.

In embodiments, the TMS is selected from the group consisting of repetitive TMS (rTMS), single pulse TMS (spTMS) and paired pulse TMS (ppTMS). In embodiments, the TMS is rTMS. In embodiments, the TMS is spTMS. In embodiments, the TMS is ppTMS.

In embodiments, the second plurality of non-invasive stimulations is administered to a left dorsolateral prefrontal cortex (DLPFC), right DLPFC, dorsal cingulate, dorsomedial prefrontal cortex, frontopolar cortex, and/or ventrolateral prefrontal cortex of the subject. In embodiments, the second plurality of non-invasive stimulations is administered to a left DLPFC of the subject. In embodiments, the second plurality of non-invasive stimulations is administered to a right DLPFC of the subject. In embodiments, the second plurality of non-invasive stimulations is administered to a dorsal cingulate of the subject. In embodiments, the second plurality of non-invasive stimulations is administered to a dorsomedial prefrontal cortex of the subject. In embodiments, the second plurality of non-invasive stimulations is administered to a frontopolar cortex of the subject. In embodiments, the second plurality of non-invasive stimulations is administered to a ventrolateral prefrontal cortex of the subject.

In embodiments, the first plurality of responses is measured via electroencephalogram (EEG), Magnetoencephalography (MEG), Functional magnetic resonance imaging (fMRI), and/or Near-infrared spectroscopy (NIRS). In embodiments, the first plurality of responses is measured via EEG. In embodiments, the first plurality of responses is measured via MEG. In embodiments, the first plurality of responses is measured via fMRI. In embodiments, the first plurality of responses is measured via NIRS.

In embodiments, the first plurality of responses is measured via EEG or fMRI concurrently with or immediately after the TMS (TMS/EEG or TMS/fMRI). In embodiments, the first plurality of responses is measured via EEG concurrently with the TMS. In embodiments, the first plurality of responses is measured via EEG immediately after the TMS. In embodiments, the first plurality of responses is measured via fMRI concurrently with the TMS. In embodiments, the first plurality of responses is measured via fMRI immediately after the TMS.

### Methods of Detection

In an aspect, there is provided a method of detecting a brain abnormality in a subject that has not been diagnosed with a clinical brain dysfunction or disorder. The method includes administering a non-invasive stimulation to a plurality of different brain regions of the subject, wherein the non-invasive stimulation to each of the plurality of different brain regions is optionally different, measuring a response to said non-invasive stimulation in each of said plurality of different brain regions thereby obtaining a measured response in each of said plurality of different brain regions; and identifying the measured response as being an abnormal response by at least applying, to the measured response, a machine learning model, thereby detecting a brain abnormality.

In an aspect, there is provided a method of detecting a brain abnormality in a subject that has not been diagnosed with a clinical brain dysfunction or disorder. The method includes administering a non-invasive stimulation to a plurality of different brain regions of the subject, wherein the non-invasive stimulation to each of the plurality of different brain regions is optionally different, measuring a response to said non-invasive stimulation in each of said plurality of different brain regions thereby obtaining a measured response in each of said plurality of different brain regions; and comparing the measured response to a control response thereby detecting a brain abnormality in the subject.

In embodiments, the measuring a response is the measurement of a timing of the response, a magnitude in response, a frequency of the response, a phase of the response, a duration of the response, and/or a presence or an absence of the response. In embodiments, the measuring a response is the measurement of a combination of one or more of a timing of the response, a magnitude in response, a frequency of the response, a phase of a response and a duration of the response. In embodiments, the measuring a response is the measurement of a combination of a timing of the response, a magnitude in response, a frequency of the response, a phase of a response, and a duration of the response. In embodiments, the variables measured (i.e., a timing of the response, a magnitude in response, a frequency of the response, a phase of the response, a duration of the response), either individually or in combination, may include measurements of the single or combined variables in one or more brain regions.

In embodiments, the measuring a response is the measurement of a timing of the response. In embodiments, the timing of the response is measured at 5-25 msecs, 25-50 msecs, 100-150 msecs, or 180 and 200 msecs following non-invasive brain stimulation. The response can be measured between 25-50 msecs (p30), 30-70 msecs (p60), 70-120 msecs (n100), 150-250 msecs (p200). In embodiments, the timing of the response is measured at 1 msec, 5 msecs, 10 msecs, 15 msecs, 25 msecs, 50 msecs, 75 msecs, 100 msecs, 150 msecs, 200 msecs, 250 msecs, 300, msecs, 400 msecs, 500 msecs, 600 msecs, 700 msecs, 800 msecs, 900 msecs, 1 sec, 1.5 secs, 2 secs, 2.5 secs, 3 secs, 3.5, secs, 4 secs, 4.5 secs, 5 secs, 5.5 secs, 6 secs, 6.5 secs, 7 secs, 7.5 secs, 8 secs, 8.5 secs, 9 secs, 9.5 secs, 10 secs, 15 secs, 20 secs, 30 secs, 40, secs, 50 secs, 1 min, 5 mins, 10 mins, 15 mins, 20 mins, 25 mins, 30 mins or any time within this range following non-invasive brain stimulation. In embodiments, the timing of the response is measured relative to the onset of the non-invasive stimulation. In embodiments, a timing of the response at 100 msec or a delay of the timing of the response (such as a delay of 10 msecs) indicates a dysfunctional brain network circuit or a brain abnormality. In embodiments, an abnormal timing of the response is determined by comparing a timing of the response in a subject against a timing of the response in a control subject. In embodiments, an abnormal timing of the response is a timing of the response that is at least 5%, 10%, 15%, 20%, 30%, 40%, 50%, 60%, 70%, 80%, 90%, 100% or more different (e.g., slower or faster) from a response measured in a control subject. In embodiments, an abnormal timing of the response is a timing of the response that falls 1.5 standard deviations or more away from the response distribution measured in a control population. In embodiments, an abnormal timing of the response is a timing of the response that is at least 5%, 10%, 15%, 20%, 30%, 40%, 50%, 60%, 70%, 80%, 90%, 100% or more different (e.g., slower or faster) from the average response measured in a control population. In embodiments, an abnormal timing of the response is a timing of the response that is at least 5%, 10%, 15%, 20%, 30%, 40%, 50%, 60%, 70%, 80%, 90%, 100% or more different (e.g., slower or faster) from the median response measured in a control population.

In embodiments, the measuring a response is the measurement of a magnitude in response. When using EEG to measure a response magnitude, the magnitude may be measured in microvolts. Thus, in embodiments, the magnitude of the response is measured in microvolts. In embodiments, a magnitude of response is a magnitude that falls within a range of about 0.2 to about 5 microvolts. In embodiments, an abnormal magnitude in response is determined by comparing a magnitude in a subject against a magnitude in a control subject. In this way, a magnitude difference may be determined. In embodiments, an abnormal magnitude in response is a magnitude that differs (e.g., positively or negatively) from a control subject magnitude by about 0.2 to about 5 microvolts or any number in this range. In embodiments, an abnormal magnitude in response is a magnitude that is at least 5%, 10%, 15%, 20%, 30%, 40%, 50%, 60%, 70%, 80%, 90%, 100% or more different (e.g., greater or smaller) from a magnitude measured in a control subject. In embodiments, an abnormal magnitude in response is a magnitude that falls 1.5 standard deviations or more away from the magnitude distribution measured in a control population. In embodiments, an abnormal magnitude in response is a magnitude that is at least 5%, 10%, 15%, 20%, 30%, 40%, 50%, 60%, 70%, 80%, 90%, 100% or more different (e.g., greater or smaller) from the average magnitude measured in a control population. In embodiments, an abnormal magnitude in response is a magnitude that is at least 5%, 10%, 15%, 20%, 30%, 40%, 50%, 60%, 70%, 80%, 90%, 100% or more different (e.g., greater or smaller) from the median magnitude measured in a control population.

EEG may also be used to determine the magnitude of a frequency of the response. In embodiments, the magnitude (e.g., amplitude in microvolts) of the frequency of the response is measured. In embodiments, the magnitude of one or more frequency ranges (e.g., delta, theta, alpha, beta gamma) is measured. In embodiments, the power in one or more frequency ranges (e.g., delta, theta, alpha, beta gamma) is measured. In embodiments, the magnitude of a dominant frequency is measured. A specific frequency resulting from the non-invasive stimulation may be referred to herein as a dominant frequency. In embodiments, the power in the dominant frequency is measured. In embodiments, the power is measured in decibels. In embodiments, an abnormal magnitude in response, e.g., magnitude of a response frequency (e.g., frequency range, dominant frequency), is determined by comparing a magnitude measured in a subject against a magnitude in a control subject. For example, the response magnitude (e.g., amplitude or power) in a one or more frequency ranges or in a dominate frequency in a subject may be compared to the response amplitude or power in a one or more frequency ranges or in a dominate frequency in a control subject or population of control subjects. In embodiments, an abnormal magnitude of a frequency of a response, measured as described herein, is a magnitude that is at least 5%, 10%, 15%, 20%, 30%, 40%, 50%, 60%, 70%, 80%, 90%, 100% or more different (e.g., greater or smaller) from a magnitude measured in a control subject. In embodiments, an abnormal magnitude of a frequency of a response, measured as described herein, is a magnitude that falls 1.5 standard deviations or more away from the magnitude distribution measured in a control population. In embodiments, an abnormal magnitude of a frequency of a response, measured as described herein, is a magnitude that is at least 5%, 10%, 15%, 20%, 30%, 40%, 50%, 60%, 70%, 80%, 90%, 100% or more different (e.g., greater or smaller) from the average magnitude measured in a control population. In embodiments, an abnormal magnitude in a frequency of a response, measured as described herein, is a magnitude that is at least 5%, 10%, 15%, 20%, 30%, 40%, 50%, 60%, 70%, 80%, 90%, 100% or more different (e.g., greater or smaller) from the median magnitude measured in a control population.

When using EEG to measure a magnitude in response, the magnitude may also be measured in pA/m in the instance that current source density analysis is used. Thus, in embodiments, the magnitude of the response is measured in pA/m. In embodiments, a magnitude in response is a magnitude that falls within a range of about 10 to about 1000 pA/m. In embodiments, an abnormal magnitude in response is determined by comparing a magnitude in a subject against a magnitude in a control subject. In this way, a magnitude difference may be determined. In embodiments, an abnormal magnitude in response is a magnitude that differs (e.g., positively or negatively) from a control subject magnitude by about 10 to about 1000 pA/m or any number in this range after source localization of current source density. In embodiments, an abnormal magnitude in response is a magnitude that is at least 5%, 10%, 15%, 20%, 30%, 40%, 50%, 60%, 70%, 80%, 90%, 100% or more different (e.g., greater or smaller) from a magnitude measured in a control subject. In embodiments, an abnormal magnitude in response is a magnitude that falls 1.5 standard deviations or more away from the magnitude distribution measured in a control population. In embodiments, an abnormal magnitude in response is a magnitude that is at least 5%, 10%, 15%, 20%, 30%, 40%, 50%, 60%, 70%, 80%, 90%, 100% or more different (e.g., greater or smaller) from the average magnitude measured in a control population. In embodiments, an abnormal magnitude in response is a magnitude that is at least 5%, 10%, 15%, 20%, 30%, 40%, 50%, 60%, 70%, 80%, 90%, 100% or more different (e.g., greater or smaller) from the median magnitude measured in a control population.

In the instance that fMRI is used to determine a magnitude in response, the magnitude is measured as a hemodynamic response, which is a response quantified as a percentage from a baseline hemodynamic measurement (e.g., 0.1 to 2.0% from baseline) where a baseline hemodynamic measurement is a measurement taken period prior (e.g., 25 msecs, 50 msecs, 100 msecs, 250 msecs, 500 msecs, 1 sec, 5 secs, 10 secs, 15 secs, 25 secs, 50 secs, 1 min, 5 mins, 10 mins, 20 mins, 30 mins, 1 hour, 24 hours, 48 hours prior) to administration of a non-invasive stimulation or during a period when non-invasive stimulation is not administered. Thus, in embodiments, the magnitude of the response is hemodynamic response that is a percent change from baseline. In embodiments, an abnormal magnitude in response is determined by comparing a magnitude in a subject against a magnitude in a control subject. In this way, a magnitude difference may be determined. In embodiments, an abnormal magnitude in response is a magnitude that differs (e.g., positively or negatively) from a control subject magnitude by about 0.1 to about 2% or any number in this range. In embodiments, an abnormal magnitude in response is a magnitude that is at least 5%, 10%, 15%, 20%, 30%, 40%, 50%, 60%, 70%, 80%, 90%, 100% or more different (e.g., greater or smaller) from a magnitude measured in a control subject. In embodiments, an abnormal magnitude in response is a magnitude that falls 1.5 standard deviations or more away from the magnitude distribution measured in a control population. In embodiments, an abnormal magnitude in response is a magnitude that is at least 5%, 10%, 15%, 20%, 30%, 40%, 50%, 60%, 70%, 80%, 90%, 100% or more different (e.g., greater or smaller) from the average magnitude measured in a control population. In embodiments, an abnormal magnitude in response is a magnitude that is at least 5%, 10%, 15%, 20%, 30%, 40%, 50%, 60%, 70%, 80%, 90%, 100% or more different (e.g., greater or smaller) from the median magnitude measured in a control population.

In embodiments, the measuring a response is the measurement of a frequency of the response. In embodiments, EEG is used to measure a frequency of a response. In embodiments, the frequencies measured are frequency ranges delta (0.5-4 Hz), theta (5-8 Hz), alpha (8-12 Hz), beta (12-30 Hz), or gamma (30-60 Hz). In embodiments, the measurement of a frequency is a measurement of a specific frequency, e.g., not a range of frequencies. A specific frequency resulting from the non-invasive stimulation may be referred to herein as a dominant frequency. In embodiments, one or more frequency ranges are measured. In embodiments, all frequencies are measured (e.g., 0.5-100 Hz). In embodiments, cross-frequency coupling (e.g., phase-phase coupling between frequencies, amplitude-amplitude coupling between frequencies, phase-amplitude coupling between frequencies) is measured. In embodiments, an abnormal frequency of a response is determined by comparing a measurement of a frequency of the response, as described herein, in a subject against a measurement of a frequency of the response, as described herein, in a control subject. For example, the response dominate frequency may be compared between a subject and a control subject or population of control subjects. In embodiments, the difference in dominant frequency between the subject and a control subject or control population is from about 0.5 to about 20 Hz. Similarly, cross-frequency coupling may be compared between a subject and a control subject or population of control subjects.

In embodiments, an abnormal frequency of a response is a frequency of a response (e.g., dominant frequency, frequency band, cross-frequency coupling), that is at least 5%, 10%, 15%, 20%, 30%, 40%, 50%, 60%, 70%, 80%, 90%, 100% or more different from a response frequency (e.g., dominant frequency, frequency band, cross-frequency coupling) measured in a control subject. In embodiments, an abnormal frequency of a response is a frequency of a response (e.g., dominant frequency, frequency band, cross-frequency coupling) that falls 1.5 standard deviations or more away from the frequency of a response (e.g., dominant frequency, frequency band, cross-frequency coupling) measured in a control population. In embodiments, an abnormal frequency of a response is a frequency of a response (e.g., dominant frequency, frequency band, cross-frequency coupling), measured as described herein, that is at least 5%, 10%, 15%, 20%, 30%, 40%, 50%, 60%, 70%, 80%, 90%, 100% or more different from the average response (e.g., dominant frequency, frequency band, cross-frequency coupling) measured in a control population. In embodiments, an abnormal frequency of a response is a frequency of a response (e.g., dominant frequency, frequency band, cross-frequency coupling) described herein, that is at least 5%, 10%, 15%, 20%, 30%, 40%, 50%, 60%, 70%, 80%, 90%, 100% or more different from the median response (e.g., dominant frequency, frequency band, cross-frequency coupling) measured in a control population.

In embodiments, the measuring a response is the measurement of a phase of the response. For example, a non-invasive stimulation may elicit a response having a specific phase. In embodiments, an abnormal phase of a response is a phase that is at least 5%, 10%, 15%, 20%, 30%, 40%, 50%, 60%, 70%, 80%, 90%, 100% or more different from a response phase measured in a control subject. In embodiments, an abnormal phase of a response is a phase that falls 1.5 standard deviations or more away from the phase of a response measured in a control population. In embodiments, an abnormal phase of a response is a phase that is at least 5%, 10%, 15%, 20%, 30%, 40%, 50%, 60%, 70%, 80%, 90%, 100% or more different from the average response phase measured in a control population. In embodiments, an abnormal phase of a response is a phase that is at least 5%, 10%, 15%, 20%, 30%, 40%, 50%, 60%, 70%, 80%, 90%, 100% or more different from the median response phase measured in a control population.

In embodiments, the measuring a response is the measurement of a duration of the response. In embodiments, the duration of the response is 2.5 msecs, 5 msecs, 10 msecs, 15 msecs, 20 msecs, 25 msecs, 30 msecs, 40 msecs, 50 msecs, 60 msecs, 70 msecs, 80 msecs, 90 msecs, 100 msecs, 150 msecs, 200 msecs, 250 msecs, 300 msecs, 350 msecs, 400 msecs, 450 msecs, 500 msecs, 550 msecs, 600 msecs, 700 msecs, 800 msecs, 900 msecs, 1 sec, 1.5 secs, 2 secs, 2.5 secs, 3 secs, 3.5, secs, 4 secs, 4.5 secs, 5 secs, 5.5 secs, 6 secs, 6.5 secs, 7 secs, 7.5 secs, 8 secs, 8.5 secs, 9 secs, 9.5 secs, or 10 secs. When EEG or MEG is used to measure a response duration, the duration of the response may be 2.5 msecs, 5 msecs, 10 msecs, 15 msecs, 20 msecs, 25 msecs, 30 msecs, 40 msecs, 50 msecs, 60 msecs, 70 msecs, 80 msecs, 90 msecs, 100 msecs, 150 msecs, 200 msecs, 250 msecs, 300 msecs, 350 msecs, 400 msecs, 450 msecs or 500 msecs. When fMRI is used to measure a response duration, the duration of the response may be 2.5 msecs, 5 msecs, 10 msecs, 15 msecs, 20 msecs, 25 msecs, 30 msecs, 40 msecs, 50 msecs, 60 msecs, 70 msecs, 80 msecs, 90 msecs, 100 msecs, 150 msecs, 200 msecs, 250 msecs, 300 msecs, 350 msecs, 400 msecs, 450 msecs, 500 msecs, 550 msecs, 600 msecs, 700 msecs, 800 msecs, 900 msecs, 1 sec, 1.5 secs, 2 secs, 2.5 secs, 3 secs, 3.5, secs, 4 secs, 4.5 secs, 5 secs, 5.5 secs, 6 secs, 6.5 secs, 7 secs, 7.5 secs, 8 secs, 8.5 secs, 9 secs, 9.5 secs, or 10 secs. In embodiments, an abnormal response duration is determined by comparing the duration of the response in a subject against a duration of the response in a control subject. In embodiments, an abnormal duration of the response is a duration that is at least 5%, 10%, 15%, 20%, 30%, 40%, 50%, 60%, 70%, 80%, 90%, 100% or more different (e.g., longer or shorter) from a response measured in a control subject. In embodiments, an abnormal duration of the response is a duration that falls 1.5 standard deviations or more away from the response distribution measured in a control population. In embodiments, an abnormal duration of the response is a duration that is at least 5%, 10%, 15%, 20%, 30%, 40%, 50%, 60%, 70%, 80%, 90%, 100% or more different (e.g., longer or shorter) from the average response measured in a control population. In embodiments, an abnormal duration of the response is a duration that is at least 5%, 10%, 15%, 20%, 30%, 40%, 50%, 60%, 70%, 80%, 90%, 100% or more different (e.g., longer or shorter) from the median response measured in a control population.

In embodiments, the measuring a response is the measurement of a presence of the response. In embodiments, the presence of a response is determined by measuring a variable as described herein (e.g., timing of the response, magnitude of the response, frequency of the response, phase of the response, duration of the response).

Alternatively, in embodiments, the presence of a response is a response, measured as described herein, that is 1.5 or more standard deviations away from baseline activity (e.g., spontaneous neural) measured prior (e.g., 25 msecs, 50 msecs, 100 msecs, 250 msecs, 500 msecs, 1 sec, 5 secs, 10 secs, 15 secs, 25 secs, 50 secs, 1 min, 5 mins, 10 mins, 20 mins, 30 mins, 1 hour, 24 hours, 48 hours prior) to administering the non-invasive stimulation. In embodiments, the presence of a response is a response, measured as described herein, that is 1.5 or more standard deviations away from baseline activity (e.g., spontaneous neural activity) measured during a period when a non-invasive stimulation has not been administered. The presence of a response, measured as described herein, may be determined using any suitable statistical method to compare the measured response against baseline activity (e.g., spontaneous neural activity) during which a non-invasive stimulation is not administered. In embodiments, the presence of a response is a response that is 1.1, 1.2, 1.3, 1.4, 1.5, 1.6, 1.7, 1.8, 1.9, 2, 3, 4, 5, 10, 15, 20, 30, 40, 50, 100, 150, 200, 250-fold or more greater than the baseline activity (e.g., spontaneous neural activity) measured. In embodiments, the presence of a response is a response that is 1.1, 1.2, 1.3, 1.4, 1.5, 1.6, 1.7, 1.8, 1.9, 2, 3, 4, 5, 10, 15, 20, 30, 40, 50, 100, 150, 200, 250-fold or more less than the baseline activity (e.g., spontaneous neural activity) measured. Other statistical methods for determining the presence of a response include, for example, Bayesian probabilities of a given magnitude of a response, or the strength of a multivariate pattern.

In embodiments, the measuring a response is the measurement of an absence of the response. In embodiments, the absence of a response is determined by measuring a variable as described herein (e.g., timing of the response, magnitude of the response, frequency of the response, phase of the response, duration of the response).

Alternatively, in embodiments, the absence of a response is a response, measured as described herein, that is less than 1.5 standard deviations away from baseline activity (e.g., spontaneous neural activity) measured prior (e.g., 25 msecs, 50 msecs, 100 msecs, 250 msecs, 500 msecs, 1 sec, 5 secs, 10 secs, 15 secs, 25 secs, 50 secs, 1 min, 5 mins, 10 mins, 20 mins, 30 mins, 1 hour, 24 hours, 48 hours prior) to administering the non-invasive stimulation. In embodiments, the absence of a response is a response, measured as described herein, that is less than 1.5 standard deviations away from the baseline activity (e.g., spontaneous neural activity) measured during a period when a non-invasive stimulation has not been administered. The absence of a response, measured as described herein, may be determined using any suitable statistical method to compare the measured response against baseline activity (e.g., spontaneous neural activity) measured during which a non-invasive stimulation is not administered. In embodiments, the absence of a response is a response that is less than 1.1-fold greater than the baseline activity (e.g., spontaneous neural activity) measured. In embodiments, the absence of a response is a response that is less than 1.1-fold less than the baseline activity (e.g., spontaneous neural activity) measured. Other statistical methods for determining the absence of a response include, for example, Bayesian probabilities of a given magnitude of a response, or the strength of a multivariate pattern.

In embodiments, the response indicates an over activity of the brain regions or an under activity of the brain regions. In embodiments, the response indicates an over activity of the brain regions. “Over activity”, as used herein, refers to neural activity that is enhanced compared to neural activity exhibited in a control subject (e.g., healthy control) in response to an identical non-invasive stimulation. Over activity of neural activity may be assessed by comparing a response, measured as described herein, in a subject to a response, measured as described herein, in a control subject. In embodiments, the response, measured as described herein, in a subject is compared against a distribution of responses, measured as described herein, from a population of control subjects. For example, over activity may be detected as a response with a higher frequency (e.g., greater power in one or more frequencies, larger amplitude in one or more frequencies), larger magnitude, longer duration, or faster timing of response. In embodiments, over activity is indicated by a response, measured as described herein, in a subject that is 1.1, 1.2, 1.3, 1.4, 1.5, 1.6, 1.7, 1.8, 1.9, 2, 3, 4, 5, 10, 15, 20, 30, 40, 50, 100, 150, 200, 250-fold or more greater than the response, measured as described herein, in a control subject (e.g., healthy control).

In embodiments, the response indicates an under activity of the brain regions. “Under activity”, as used herein, refers to neural activity that is reduced compared to neural activity exhibited in a control subject in response to an identical non-invasive stimulation. Under activity of neural activity may be assessed by comparing a response, measured as described herein, in a subject to a response, measured as described herein, in a control subject (e.g., healthy control). In embodiments, the response, measured as described herein, in a subject is compared against a distribution of responses, measured as described herein, from a population of control subjects. For example, under activity may be detected as a response with a lower frequency (e.g., less power in one or more frequencies, smaller amplitude in one or more frequencies), smaller magnitude, shorter duration, or slower timing of response. In embodiments, under activity is indicated by a response, measured as described herein, in a subject that is 1.1, 1.2, 1.3, 1.4, 1.5, 1.6, 1.7, 1.8, 1.9, 2, 3, 4, 5, 10, 15, 20, 30, 40, 50, 100, 150, 200, 250-fold or more less than the response, measured as described herein, in a control subject (e.g., healthy control).

In embodiments, the method further includes treating the subject with repetitive transcranial magnetic stimulation (rTMS) where an over activity is measured. In embodiments, the method further includes treating the subject with rTMS where an under activity is measured.

In embodiments, each of plurality of different brain regions of the subject is selected from the group consisting of frontal cortex, temporal cortex, parietal cortex, occipital cortex, hippocampus, amygdala, striatum or brainstem, and subregions thereof.

In embodiments, the non-invasive stimulation is administered to a left dorsolateral prefrontal cortex (DLPFC), right DLPFC, dorsal cingulate, dorsomedial prefrontal cortex, frontopolar cortex, and/or ventrolateral prefrontal cortex of the subject. In embodiments, the non-invasive stimulation is administered to a left DLPFC of the subject. In embodiments, the non-invasive stimulation is administered to a right DLPFC of the subject. In embodiments, the non-invasive stimulation is administered to a dorsal cingulate of the subject. In embodiments, the non-invasive stimulation is administered to a dorsomedial prefrontal cortex of the subject. In embodiments, the non-invasive stimulation is administered to a frontopolar cortex of the subject. In embodiments, the non-invasive stimulation is administered to a ventrolateral prefrontal cortex of the subject.

In embodiments, the non-invasive stimulation to each of the plurality of different brain regions is different. In embodiments, the non-invasive stimulation to each of the plurality of different brain regions is same.

In embodiments, the non-invasive stimulation is transcranial magnetic stimulation (TMS), focused ultrasound stimulation, transcranial direct current or transcranial alternating current stimulation. In embodiments, the non-invasive stimulation is TMS. In embodiments, the non-invasive stimulation is focused ultrasound stimulation. In embodiments, the non-invasive stimulation is transcranial direct current stimulation (tDCS). In embodiments, the non-invasive stimulation is transcranial alternating current stimulation (tACS). In embodiments, the non-invasive stimulation is one or more (e.g., 1, 2, 3 or 4) selected from the group consisting of transcranial magnetic stimulation (TMS), focused ultrasound stimulation, transcranial direct current and transcranial alternating current stimulation.

In embodiments, TMS is selected from the group consisting of rTMS, single pulse TMS (spTMS) and paired pulse TMS (ppTMS). In embodiments, TMS is rTMS. In embodiments, TMS is spTMS. In embodiments, TMS is ppTMS.

In embodiments, the response is measured via electroencephalogram (EEG), Magnetoencephalography (MEG), Functional magnetic resonance imaging (fMRI), and/or Near-infrared spectroscopy (NIRS). In embodiments, the response is measured via EEG. In embodiments, the response is measured via MEG. In embodiments, the response is measured via fMRI. In embodiments, the response is measured via NIRS.

In embodiments, the response is measured via EEG or fMRI concurrently with or immediately after the TMS (TMS/EEG or TMS/fMRI). In embodiments, the response is measured via EEG concurrently with the TMS. In embodiments, the response is measured via EEG immediately after the TMS. In embodiments, the response is measured via fMRI concurrently with the TMS. In embodiments, the response is measured via fMRI immediately after the TMS.

In embodiments, the machine learning model includes a neural network, a regression model, an instance-based model, a regularization model, a decision tree, a Bayesian model, a clustering model, an associative model, a deep learning model, a dimensionality reduction model, and/or an ensemble model. In embodiments, the machine learning model includes a neural network. In embodiments, the machine learning model includes a regression model. In embodiments, the machine learning model includes an instance-based model. In embodiments, the machine learning model includes a regularization model. In embodiments, the machine learning model includes a decision tree. In embodiments, the machine learning model includes a Bayesian model. In embodiments, the machine learning model includes a clustering model. In embodiments, the machine learning model includes an associative model. In embodiments, the machine learning model includes a deep learning model. In embodiments, the machine learning model includes a dimensionality reduction model. In embodiments, the machine learning model includes an ensemble model.

In embodiments, the machine learning model may cluster one or more or a plurality or combination of variables by at least applying one or more clustering techniques including, for example, density-based clustering (e.g., density-based spatial clustering of applications with noise (DBSCAN), hierarchical density-based spatial clustering of applications with noise (HDBSCAN)) and centroid-based clustering (e.g., k-nearest neighbor clustering, k-means clustering). In doing so, the machine learning model may generate clusters that group same and/or similar variables or combinations of variable, which may be associated with responses having the same and/or similar classification (e.g., normal, abnormal).

In embodiments, the machine learning model may apply one or more dimensionality reduction techniques to reduce the dimensionality of the measured response. For instance, when the measured response is defined by a plurality of variables or a combination of variable, the machine learning model may apply a dimensionality reduction technique such as random projection, robust principal component analysis (RPCA), t-distributed stochastic neighbor embedding (t-SNE), and/or the like. Applying the dimensionality reduction technique may reduce the dimensionality of the measured response, as described by a plurality of variables or combination of variable, from a high-dimensionality feature space to a lower-dimensionality feature space, thereby facilitating a subsequent assignment of a measured response to one or more clusters of measured responses having known classifications (e.g., normal or abnormal).

In embodiments, the machine learning model will assign, based at least on one or more or a plurality of variables (e.g., timing of the response, magnitude in response, frequency of the response, phase of the response, duration of the response) the measured response to one of a plurality of clusters that group measured responses having known a classification (e.g., normal or abnormal). In embodiments, the machine learning model may have generated, by applying one or more density-based clustering algorithms, a plurality of clusters that group variables having known classifications (e.g., normal or abnormal).

In embodiments, the machine learning model is trained to identify one or more abnormal responses.

In embodiments, the method further includes training, based at least on training data, the machine learning model, the training data including a plurality of abnormal responses and/or normal responses, and the machine learning model being trained to differentiate between the abnormal responses and the normal responses.

In embodiments, the training of a machine learning model is supervised. In this case, the training data used for training the model is linked to a known condition (e.g., presence or absence of a disease, disorder, dysfunction (e.g., brain dysfunction), abnormality (e.g., brain abnormality)). For example, the training data includes data linked to a control subject or population of control subjects, or a subject or population of subjects known to suffer from a disease, condition, or disorder (e.g., brain dysfunction, brain abnormality). Thus, the model may be trained to classify (e.g., cluster) the data according to normal and abnormal responses. In this way, the model, when presented with data (e.g., measured responses defined by variables as described herein) of unknown origin, may associate the novel data with the known classified (e.g., clustered) normal or abnormal data. Associate of novel data with a cluster may be accomplished by the model through the use of, for example, centroid-based clustering techniques, such as k-means clustering and/or the like.

Alternatively, in embodiments, the training of a machine learning model is unsupervised. In this case, the training data used for training the model is not linked to a known condition (e.g., presence or absence of a disease, disorder, dysfunction (e.g., brain dysfunction), abnormality (e.g., brain abnormality)). In this way, the model, when presented with data (e.g., measured responses defined by variables as described herein) of unknown origin, may associate the novel data with a cluster generated by the unsupervised data. As such, the model may classify the novel data as part of an existing cluster generated by the unsupervised training data or as part of a cluster different from the existing clusters or an outlier. In this way a response may be determined to be abnormal or normal. Associate of novel data with a cluster may be accomplished by the model through the use of, for example, centroid-based clustering techniques, such as k-means clustering and/or the like.

In embodiments, the training data includes a plurality of variables associated with abnormal responses and/or normal responses, and wherein the plurality of variables includes a timing of the response, a magnitude in response, a frequency of the response, a phase of the response, a duration of the response, and/or a presence or an absence of the response.

In embodiments, the plurality of variables includes a timing of the response. In embodiments, the timing of the response occurs at 25-50 msecs, 100-150 msecs, or 180 and 200 msecs following non-invasive brain stimulation. In embodiments, the timing of the response occurs between 25-50 msecs (p30), 30-70 msecs (p60), 70-120 msecs (n100), 150-250 msecs (p200). In embodiments, the timing of the response occurs at 1 msec, 5 msecs, 10 msecs, 15 msecs, 25 msecs, 50 msecs, 75 msecs, 100 msecs, 150 msecs, 200 msecs, 250 msecs, 300, msecs, 400 msecs, 500 msecs, 600 msecs, 700 msecs, 800 msecs, 900 msecs, 1 sec, 1.5 secs, 2 secs, 2.5 secs, 3 secs, 3.5, secs, 4 secs, 4.5 secs, 5 secs, 5.5 secs, 6 secs, 6.5 secs, 7 secs, 7.5 secs, 8 secs, 8.5 secs, 9 secs, 9.5 secs, 10 secs, 15 secs, 20 secs, 30 secs, 40, secs, 50 secs, 1 min, 5 mins, 10 mins, 15 mins, 20 mins, 25 mins, 30 mins or any time within this range following non-invasive brain stimulation. In embodiments, the timing of the response is determined relative to the onset of the non-invasive stimulation.

In embodiments, the plurality of variables includes a magnitude in response. In embodiments, the magnitude of the response is measured in microvolts. In embodiments, a magnitude in response falls within a range of about 0.2 to about 5 microvolts. In embodiments, a magnitude in response is measured in pA/m. In embodiments, a magnitude in response is a magnitude that falls within a range of about 10 to about 1000 pA/m. In embodiments, the magnitude in response is hemodynamic response that is measured as a percent change from a baseline hemodynamic measure taken during a period in which no non-invasive stimulation was delivered. In embodiments, the magnitude in response is the magnitude of the frequency of the response. In embodiments, the magnitude in response is the magnitude in one or more frequency ranges (e.g., delta, theta, alpha, beta, gamma). In embodiments, the magnitude in response is the power in one or more frequency ranges (e.g., delta, theta, alpha, beta, gamma). In embodiments, the magnitude in response is the magnitude in a dominant frequency. In embodiments, the magnitude in response is the power in the dominant frequency. In embodiments, the magnitude in response is measured in decibels.

In embodiments, the plurality of variables includes a frequency of the response, measured as described herein. Thus, in embodiments, the frequency of response is a dominant frequency or a frequency band (e.g., delta, theta, alpha, beta, gamma). In embodiments, the frequency of response is the cross-frequency coupling (e.g., phase-phase coupling between frequencies, amplitude-amplitude coupling between frequencies, phase-amplitude coupling between frequencies).

In embodiments, the plurality of variables includes a phase of the response.

In embodiments, the plurality of variables includes a duration of the response. In embodiments, the duration of the response includes 2.5 msecs, 5 msecs, 10 msecs, 15 msecs, 20 msecs, 25 msecs, 30 msecs, 40 msecs, 50 msecs, 60 msecs, 70 msecs, 80 msecs, 90 msecs, 100 msecs, 150 msecs, 200 msecs, 250 msecs, 300 msecs, 350 msecs, 400 msecs, 450 msecs, 500 msecs, 550 msecs, 600 msecs, 700 msecs, 800 msecs, 900 msecs, 1 sec, 1.5 secs, 2 secs, 2.5 secs, 3 secs, 3.5, secs, 4 secs, 4.5 secs, 5 secs, 5.5 secs, 6 secs, 6.5 secs, 7 secs, 7.5 secs, 8 secs, 8.5 secs, 9 secs, 9.5 secs, 10 secs. In embodiments, when EEG or MEG is used to measure a response duration, the duration of the response is 2.5 msecs, 5 msecs, 10 msecs, 15 msecs, 20 msecs, 25 msecs, 30 msecs, 40 msecs, 50 msecs, 60 msecs, 70 msecs, 80 msecs, 90 msecs, 100 msecs, 150 msecs, 200 msecs, 250 msecs, 300 msecs, 350 msecs, 400 msecs, 450 msecs or 500 msecs. In embodiments, when fMRI is used to measure a response duration, the duration of the response is 2.5 msecs, 5 msecs, 10 msecs, 15 msecs, 20 msecs, 25 msecs, 30 msecs, 40 msecs, 50 msecs, 60 msecs, 70 msecs, 80 msecs, 90 msecs, 100 msecs, 150 msecs, 200 msecs, 250 msecs, 300 msecs, 350 msecs, 400 msecs, 450 msecs, 500 msecs, 550 msecs, 600 msecs, 700 msecs, 800 msecs, 900 msecs, 1 sec, 1.5 secs, 2 secs, 2.5 secs, 3 secs, 3.5, secs, 4 secs, 4.5 secs, 5 secs, 5.5 secs, 6 secs, 6.5 secs, 7 secs, 7.5 secs, 8 secs, 8.5 secs, 9 secs, 9.5 secs, or 10 secs.

In embodiments, the plurality of variables includes a presence of the response. A presence of a response is as described supra.

In embodiments, the plurality of variables includes an absence of the response. An absence of a response is as described supra.

In embodiments, the disclosures herewith provide a method to determine where in the brain of an individual with a disorder of the central nervous system abnormalities in brain function are elicited through non-invasive brain stimulation. The purpose of identifying these locations, as well as their particular signatures (i.e. the nature of the abnormality) is that this can then guide neurostimulation interventions that normalize the identified abnormality. The method therefore can be used to “diagnose” brain abnormalities in an actionable way (for example, by virtue of them being identified through non-invasive brain stimulation and that repetitive non-invasive brain stimulation can cause lasting changes in the function of brain circuitry it targets). As such, this method of identifying and remediating evoked brain abnormalities does not entirely or substantially rely on a specific diagnosis of a psychiatric or neurological disorder (or condition such as chronic pain). Rather, it allows the physicians to focus on the brain of their individual patient, and thus form the basis of a personalized neurostimulation therapy.

In embodiments, a method according to the disclosures can employ the following steps: identify dysfunction in causal signal flow (either excessive or insufficient) in the brains of individual patients by mapping their causal connectome, and then use this information to guide an adaptable circuit remediation treatment. In embodiments, a causal connectome refers to a stimulation-evoked brain response. Thus, in embodiments, causal connectome mapping can be defined as determining the causal (e.g. stimulation-evoked) brain response to stimulation at one or multiple brain regions in an individual's brain. Notably, this is very different with a one-size-fits-all approach currently being used for neurostimulation treatment (e.g. rTMS for depression), which guides treatment to a particular location in a similar way for all individuals who carry a general diagnosis.

In embodiments, a method according to the disclosures can have Phases (or Procedures) 1 and 2 as described below.

Phase 1 (or Procedure 1): Causal connectomic mapping. In embodiments, this phase can be done by using the combination of single pulse TMS (spTMS) as the neurostimulation method for probing evoked brain responses and EEG as the method for measuring them. This embodiment is just one form that this method can take and other types of non-invasive stimulation and measurement means can be employed. For example, the non-invasive stimulation can include any neurostimulation method that evokes measurable brain responses, which can be indexed against a distribution of responses in the brains of healthy individuals, can be used. This includes, but is not limited to, methods such as transcranial magnetic stimulation (TMS), focused ultrasound (fUS), transcranial alternating or direct current (tACS, tDCS) as the currently available techniques. Measurement of brain functional readout is likewise flexible, including functional magnetic resonance imaging (fMRI), electroencephalography (EEG), magnetoencephalography (MEG), and functional near infrared spectroscopy (fNIRS) as the currently available methods.

In embodiments, a method according to the disclosures can robustly map brain responses to non-invasive stimulation (e.g. spTMS stimulation) at a plurality (i.e. one or more) of sites across the cortex of an individual in a measurement, e.g. a single EEG session. In embodiments, the number of sites (i.e. target sites or targets) in the individual's brain (or cortex) that are non-invasively stimulated and the responses thereto are measured can be at least two or more, three or more, four or more, five or more, ten or more, fifteen or more, twenty or more, twenty five or more, thirty or more, thirty five or more, forty or more, forty five or more, fifty or more, sixty or more, seventy or more, eighty or more, ninety or more, a hundred of more, or any intervening number or more. In embodiments, the number of non-invasive stimulation-positioning and optimization of the stimulation and analytic methods can be increased to 50-100 targets or more, thus partially or substantially blanketing the cortex. In embodiments, it can also be applied in a more focal way in a particular zone of the brain with any number of stimulation sites. Processing of the data as they are acquired during the performance of the method can yield in real-time the high-density causal map of the individual cortex to date, including its spatio-temporal circuit dynamics. In embodiments, the brain regions subject to non-invasive stimulation and response measurement may include any regions of the individual's brain, in particular, any area of cortex, including but not limited to, frontal cortex, temporal cortex, parietal cortex, occipital cortex, hippocampus, amygdala, striatum or brainstem, any subregions thereof, and any combinations of the foregoing-listed regions.

In embodiments, causal connectomes of a plurality (i.e. one or more) of healthy individuals can be used to create a reference data set, to which individual patient causal connectomes can be compared using methods such as multivariate pattern classification. This can identify for each patient where responses to stimulation were excessive or inadequate. Phase 1 (or Procedure 1) therefore can yield an unprecedented source of new information about normative patterns of causal connectomic cortical organization in individual patients, and establish a powerful method for characterizing abnormalities in causal cortical signal flow in each of the patients.

Phase 2 (or Procedure 2): Remediation of dysfunctional circuits. In embodiments, there can be generalizable “circuit plasticity rules” governing the relationship between the nature of causal disruption of evoked brain responses and the plasticity induction method (e.g. rTMS protocol) required for restoring durable normative circuit functioning. In embodiments, the circuit plasticity rules may be set up based on a reliable relationship between the way plasticity is induced and the kind of effects it has on whatever brain system is targeted by it, independent of the specific brain target. These rules can be employed to match a particular plasticity induction method with the identified abnormality. For instance, in embodiments, patients with an excessive inhibitory rebound to dorsolateral prefrontal stimulation may benefit from an excitatory rTMS intervention that reduces this response. By tracking change in the spTMS-evoked neurophysiological signals that are excessive or insufficient in that individual, an optimal plasticity inducing rTMS protocol can be identified and applied until lasting circuit remediation is achieved (or until it is evident that the circuit dysfunction cannot be repaired). Empirically-validated individualized treatment plans can then identify which abnormalities are impactful (or effective) for symptoms and behavior, and which, despite being causal in terms of signal flow, can have less impact when remediated. There can also be a case that each patient is characterized by a unique set of abnormalities, or alternatively that a limited set of abnormality patterns exist. In either case, characterization of the individual's brain circuit can be mapped (or determined). Circuit remediation based on the personalized mapping of the individual patient's brain circuit can yield insights about abnormalities in the patient's capacity for plasticity in specific circuits, and elucidate the causal relevance of abnormalities in these circuits for symptoms and behavior. Using the principles of associative plasticity, a treater can weaken or strengthen specific pathways through the asynchronous stimulation of pairs of brain regions with, e.g. separate TMS coils.

The method provided herewith can effectively intervene at the circuit level in psychiatry, neurology and chronic pain and it can be further effective by combining neurostimulation with manipulations of neuronal activity using pharmacological or behavioral tools (e.g. dopaminergic agonists, nicotine, etc.). Therefore, the disclosures herewith provide systems and methods that successfully establish a platform for circuit plasticity interventions in humans, guided by neurophysiological signals measured in real-time.

Remediation or optimization of a treatment protocol for an individual patient may be enacted in real time via monitoring of the non-invasive brain stimulation evoked response, for a closed-loop individualized treatment. In embodiments, remediation or optimization of a treatment protocol for an individual patient may also include creation, development, customization and/or alteration of a treatment protocol suitable to the individual patient. In embodiments, a machine learning protocol can be adapted to analyze characteristics of a non-invasive brain stimulation evoked response and alter the treatment as it continues. In embodiments, remediation or optimization to treatment protocols occur in real time, at a following treatment session (e.g., within hours, within a single day, within days, within weeks). Furthermore, monitoring a non-invasive brain stimulation evoked response may occur following an initial course of treatment as a disease monitoring, prophylactic of diagnostic method. In embodiments, monitoring of a non-invasive brain stimulation evoked response may occur about one to four weeks, one month, two months, three months, 6 months, one year, or more following a successfully complete course of treatment.

In embodiments, Phase 2 (or Procedure 2) for remediating dysfunctional circuits may include one or more of the following and any modifications or revisions thereof:
1) Selecting the optimal site or sites to target for repetitive stimulation that is aimed at normalizing the abnormality observed (and which guided targeting of therapeutic stimulation)
2) Optimization of parameters such as coil angle, pulse width, pulse height, pulse shape etc. based on their evocation of a brain response that is being targeted for remediation
3) Selection of the best repetitive stimulation protocol that best alters brain function in the direction of normalization of the observed abnormality (including frequency, intensity and pattern of stimulation)
4) Use of a task or other method to manipulate a state of the brain so that it best modifies the ability of the repetitive stimulation to lead to normalization of the observed abnormality
5) Use of a pharmacological agent that can do similarly
6) Use of a different (or multiple) neurostimulation modalities
7) Targeting of multiple regions (e.g. that form a network or are simply interconnected), including targeting the input to or output from a region found to be abnormal when stimulated during causal connectivity mapping.

In embodiments, remediating dysfunctional circuits may include selection of a plurality (i.e. one or more) of sites for non-invasive stimulation on the patient's brain wherein the selected sites are identical or a subset of the sites stimulated during the mapping phase (or Phase 1). Therefore, after the mapping phase, the target sites for treatment can be selected based on the mapped brain circuit of the patient and the circuit plasticity rules. In embodiments, at least some of the sites selected for stimulation during the remediating phase (Phase 2) can be different from those stimulated during the mapping phase (Phase 1). In embodiments, the number of sites selected for stimulation during the remediating phase (Phase 2) can be more, identical or less than the number of sites stimulated during the mapping phase (Phase 1).

The systems and methods according to the disclosures herewith can generally be applicable to any condition affecting an individual's mental condition, in particular, the central nervous system that leads to changes in evoked brain responses through non-invasive neurostimulation. In embodiments, this can includes all psychiatric disorders, many neurological disorders (e.g. stroke, Parkinson's, Alzheimer's) and other conditions such as chronic pain.

In embodiments, TMS is used for non-invasive stimulation during a mapping phase (procedure) and/or treatment phase (procedure). TMS is a non-invasive technique that typically involves placing a coil near the patient's head to depolarize or hyperpolarize neurons of the brain. In particular, TMS uses electromagnetic induction to induce neuronal electrical currents using a rapidly changing magnetic field. A changing magnetic field leads to changing electrical currents by causing transient shifts in ions across neuron cell membranes. The brain region underneath the TMS coil is the primary target for the TMS effect, with further distant areas of the brain being impacted through the initial impulse delivered to the targeted region under the coil. TMS techniques typically act on a volume of brain tissue that is approximately two to three centimeters in diameter. TMS methods can include repetitive TMS (rTMS), single pulse TMS (spTMS), or paired pulse TMS (ppTMS).

In an example treatment protocol, daily rTMS induces long-lasting cortical neuromodulatory effects across broadly distributed regions. These effects are temporally and spatially removed from the onset and location of stimulation, but are highly predictive of clinical outcome. Mechanistically, non-invasive and invasive studies suggest that rTMS induces a reduction in early, local evoked gamma power and an early excitatory electrophysiological response, and an increase in later alpha power and slower inhibitory electrophysiological responses, suggesting a lasting alteration in the excitability of brain networks and altered interaction between brain regions and networks.

Treatment protocols for each type of TMS vary in duration, time course, pulse sequence, magnitude of stimulation and area of stimulation. Course of treatment can vary in duration from about one day, two days, three days, four days, five days, six days, seven days, one week, two weeks, three weeks, four weeks, five weeks, six weeks, seven weeks, eight weeks, or more. Frequency of TMS stimulation can vary (e.g., about 10, 20, or 30 Hz). TMS stimulation can be 1 Hz TMS, 3 Hz TMS, 5 Hz TMS, 7 Hz TMS, 10 Hz TMS, 15 Hz TMS, 20 Hz TMS, 25 Hz TMS, 30 Hz TMS or intermittent theta burst TMS. Paired pulse TMS can be administered at a time offset of about 10 milliseconds (msecs or ms), 20 msecs, 30 msecs, 40 msecs, 50 msecs, 100 msecs, 150 msecs, 200 msecs, 250 msecs, 300 msecs, or more. In embodiments, TMS can be administered to the right or left prefrontal cortices (e.g., left dorsolateral prefrontal cortex (DLPFC), right DLPFC, dorsal cingulate, dorsomedial prefrontal cortex, frontopolar cortex, ventrolateral prefrontal cortex.).

In embodiments, non-invasive brain stimulation evoked response can be measured via electroencephalogram (EEG), Magnetoencephalography (MEG), Functional magnetic resonance imaging (fMRI), or Near-infrared spectroscopy (NIRS). In embodiments, a magnitude of a non-invasive brain stimulation evoked response is measured at 25-50 msecs, 100-150 msecs, or 180 and 200 msec following non-invasive brain stimulation. The TMS evoked response can be measured between 25-50 msecs (p30), 30-70 msecs (p60), 70-120 msecs (n100), 150-250 msecs (p200). Alternatively, the TMS evoked response can be measured on the amplitude of oscillations at theta (5-8 Hz), alpha (8-12 Hz), beta (12-30 Hz), or gamma (30-60 Hz) within the first second after a TMS pulse.

In embodiments, TMS can achieve neurostimulation at sub-millisecond precision by application of a brief and focal magnetic field, which transiently excites the cortical region underlying the TMS coil. This perturbation can be followed by an inhibitory rebound within the circuit, which can last, e.g. for several hundred milliseconds, and can be followed by re-establishment and synchronization of oscillatory rhythms. The effects of single TMS pulses (spTMS) can be imaged at the temporal scale of neurons in cortex using concurrent EEG, or it can be imaged on a fine-grain spatial resolution anywhere in the brain using concurrent functional magnetic resonance imaging (fMRI). By imaging the effects of spTMS-mediated targeted circuit perturbation, the insight about causal signal flow through these regions can be obtained. Doing so for a number of target locations across cortex (e.g. two or more) can thereby map a “causal connectome” at the individual patient level. Application of patterned repetitive TMS (rTMS) pulses can lead to durable plastic changes in circuit function, either increasing or decreasing efficacy of causal communication, the effects of which can be tracked with concurrent EEG or fMRI.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of various embodiments, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### EXAMPLES

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1

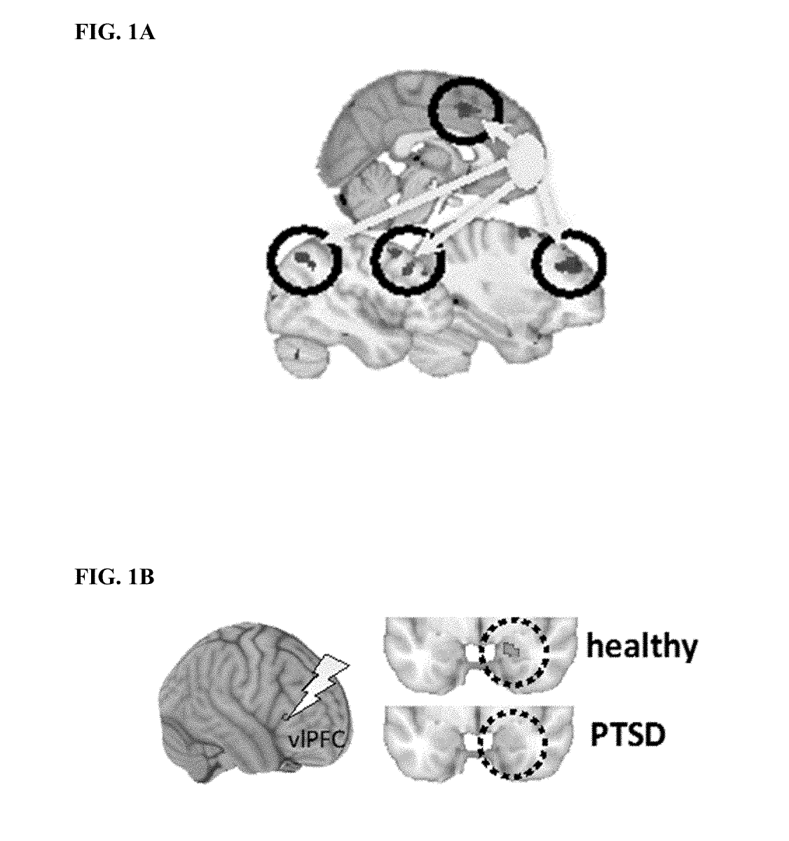

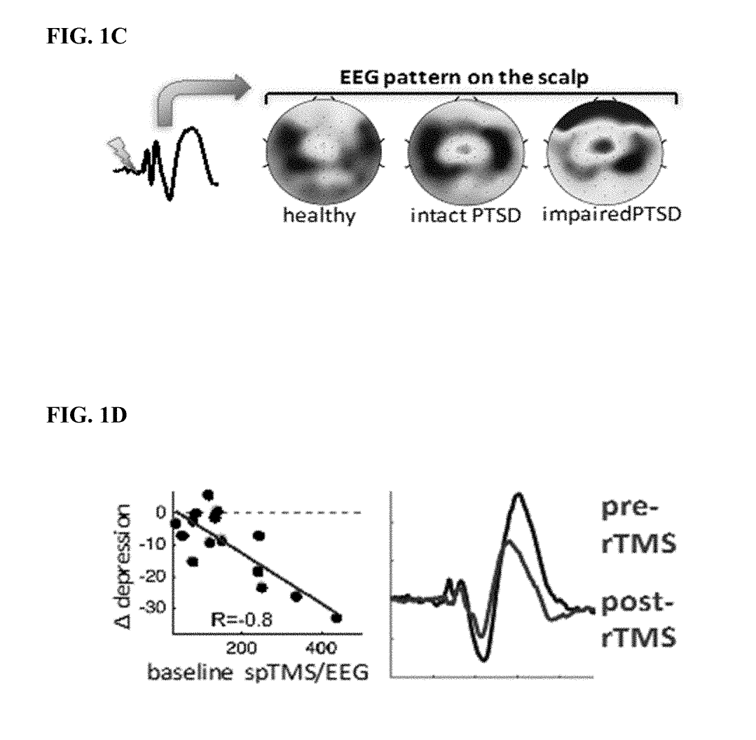

Discovering causal human brain network relationships: A method employing spTMS/fMRI was used to establish the causal mechanisms underlying the balance between brain regions implicated in attending and responding to external task demands and those implicated in internally-focused mentation, which typically decrease activity during attention-demanding tasks. The method stimulated two regions in the dorsolateral prefrontal cortex (DLPFC) that activate during tasks. However, in the tested subject, only one of these (the posterior DLPFC (pDLPFC)) actually exerted a causal and inhibitory effect on the medial prefrontal cortex, which typically deactivates in attention-demanding tasks (FIG. 1A). This demonstrates that causal influence may not be uniform amongst regions that display similar activation profiles during typical observational imaging studies. The method went ahead to examine whether a direct TMS-accessible cortical pathway exists for regulating activity in the amygdala, a subcortical hub for a range of emotional functions, which is overactive in mood and anxiety disorders. The concurrent spTMS/fMRI was used to stimulate a putative amygdala-controlling site in the ventrolateral prefrontal cortex (vlPFC). VLPFC activation with TMS resulted in inhibition of the amygdala (FIG. 1B)—an effect not seen in patients with PTSD. Furthermore, disruption of this region in healthy individuals resulted in effects on behavior that mirrored those reported in PTSD. These data establish for the first time a causal cortical pathway for amygdala control in humans, as well as its clinical relevance. Thus, TMS can reveal causal connectomic relationships that could not be discovered through conventional neuroimaging alone, yielding insights about both basic brain processes and mechanisms of illness, thus informing the development of novel therapeutics.

### Example 2

Using neurobiology to overcome diagnostic heterogeneity: Using current clinical criteria, there are a shocking 636,120 ways to meet criteria for PTSD. To establish whether neurobiological phenotypes with clinical relevance can be objectively identified at the individual patient level within the broader traditional symptom-based clinical definition, the inventors conducted a neurobehavioral circuit-level characterization of PTSD that focused on core deficits in memory and resting-state network connectivity. A quarter of PTSD patients displayed both aberrant connectivity across major large-scale resting networks and impaired verbal memory. This network/memory-impairment phenotype was replicated in a separate PTSD sample despite differences in clinical diagnostic criteria, gender breakdown, clinical population, and acquisition site and methods. Across patients with or without this phenotype there was no difference in any clinical symptoms that are used to diagnose and categorize the disorder. However, the network/memory phenotype robustly predicted outcome to exposure-based psychotherapy, the best-validated treatment for PTSD, wherein network/memory-impaired patients were profoundly treatment-resistant (while those without the impairment largely remitted). Using spTMS/EEG the inventors identified specific causal circuit dysfunction that contributed to this phenotype's connectomic abnormalities—implicating an excessive putative inhibitory rebound to stimulation of the pDLPFC (FIG. 1C). Notably, spTMS stimulation to the same region increases cognitive network interactions in healthy individuals (FIG. 1A). Thus, by identifying a distinct “cogno-connectomic” form of PTSD with specific abnormalities in network architecture, causal prefrontal circuit dynamics, and treatment resistance, these findings demonstrate a novel and unified neuroscientific method for mechanistically disentangling the clinical heterogeneity of a notoriously ill-defined psychiatric entity. The term “cogno-connectomic form” of PTSD may refer to a form of PTSD characterized by both specific cognitive impairments and specific network connectivity abnormalities. These findings also provide a transition from a descriptive approach (e.g. resting fMRI, behavior) to a causal one (e.g. pDLPFC inhibitory rebound abnormalities evident by spTMS/EEG), around which treatment can be developed (by quantifying and tracking changes in causal neurophysiological processes).

### Example 3

Individual-level causal circuit interrogation: The inventors also used spTMS/EEG to uncover the causal circuit mechanisms of rTMS for depression. It was found that clinical response was greatest for those patients who at baseline showed a larger inhibitory rebound to spTMS/EEG stimulation of the DLPFC target later used for rTMS treatment (FIG. 1D). Moreover, treatment with what has been argued to be a potentiating rTMS protocol resulted in blunting of the same inhibitory rebound signal (FIG. 1D), suggesting that an overall increase in DLPFC excitability may arise from dampening of its inhibitory break. Thus, it is possible to characterize the causal strength of a particular network or region, use this information to guide where and how rTMS may be used to remediate circuit abnormalities, and then track the progress of this remediation. To harness the potential of spTMS/EEG, the inventors developed the automated artifact rejection method for these data—a process that has previously required painstaking work by a human operator. As a consequence, the method herewith can rapidly analyze in near real-time large amounts of spTMS/EEG data, such as would be created by causal connectomic mapping of patients across dozens of cortical stimulation targets in a single assessment session.

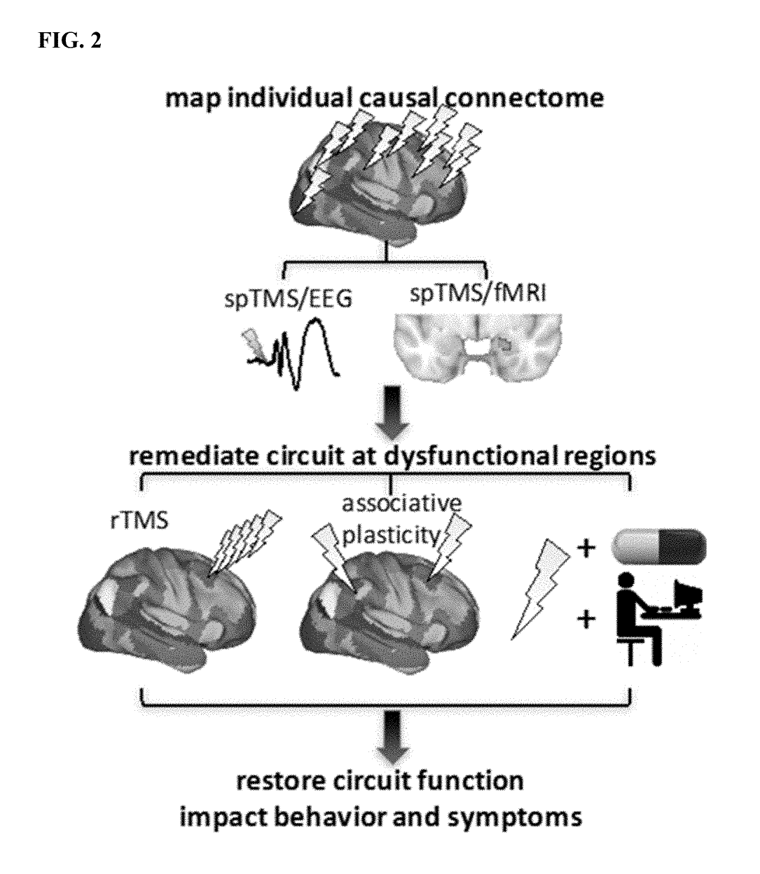

Personalized Neurostimulation: An Effective Approach to Mental Illness—The method according to some embodiments relies on identifying dysfunction in causal signal flow (either excessive or insufficient) in the brains of individual patients by mapping their causal connectome, and then using this information to guide an adaptable circuit remediation treatment (FIG. 2) by adopting the following Phases (or Procedures) 1 and 2.

Phase 1 (Procedure 1): Causal Connectomic Mapping.

The Phase 1 can robustly map responses to spTMS stimulation at 25 sites across the cortex of an individual in a single EEG session. With the aid of robotic TMS-positioning and optimization of our stimulation and analytic methods, this can be increased to 50-100 targets, thus partially or substantially blanketing the cortex. Processing of these data as they are acquired can yield in real-time the high density causal map of the human cortex to date, including its spatio-temporal circuit dynamics. Causal connectomes of many healthy individuals can be used to create a reference data set, to which individual patient causal connectomes can be compared using multivariate pattern classification methods. This can identify for each patient where responses to stimulation were excessive or inadequate. Alternatively or in combination, a similar but more targeted approach at fewer sites during concurrent spTMS/fMRI can be performed to examine causal influence of cortical regions on certain subcortical nodes such as the amygdala. The analyses of resting-state fMRI connectivity data already demonstrated that conventional neuroimaging data can robustly identify individual-level connectivity signatures.

Phase 2: Remediation of Dysfunctional Circuits.

There can be generalizable “circuit plasticity rules” governing the relationship between the nature of causal disruption of evoked EEG or fMRI responses and the rTMS protocol required for restoring durable normative circuit functioning. In embodiments, patients with the excessive inhibitory rebound to pDLPFC stimulation in FIG. 1C can benefit from an excitatory rTMS intervention that reduces this response, such as in FIG. 1D. By tracking change in the spTMS-evoked neurophysiological signals that are excessive or insufficient in that individual, an optimal plasticity-inducing rTMS protocol can be identified and applied until lasting circuit remediation is achieved (or until it is evident that the circuit dysfunction cannot be repaired). Empirically-validated individualized treatment plans can then identify which abnormalities can be impactful for symptoms and behavior, and which, despite being causal in terms of signal flow, have less impact when remediated. It can also be the case that each patient is characterized by a unique set of abnormalities, or alternatively that a limited set of abnormality patterns exist. In either case, characterization of the individual can be done. Through these procedures, it can provide meaningful insights about fundamental abnormalities in patients' capacity for plasticity in specific circuits, elucidation on the causal relevance of abnormalities in these circuits for symptoms and behavior as well as achievement of personalized and optimized circuit remediation for patients.

Beyond a circumscribed focus on remediating dysfunction in individual regions by targeting them with rTMS, the methods and systems of the disclosures can also establish how particular pathways that comprise limbs of a circuit can be remediated. Using the principles of associative plasticity, one can weaken or strengthen specific pathways through the asynchronous stimulation of pairs of brain regions with separate TMS coils (FIG. 2). This targeting of nodes with rTMS can work better for certain causal connectomic abnormalities, such as anatomically-distributed changes in longer-latency elements of the response to spTMS stimulation. In embodiments, associative plasticity methods can be better suited for other abnormalities, such as fast-latency focal alterations in putative excitatory responses to spTMS.

In embodiments, increased specificity for which types of neurons undergo therapeutic plasticity can also be achieved by combining TMS with manipulations of neuronal activity using pharmacological or behavioral tools.

### BIBLIOGRAPHY

Roehrig C: Mental Disorders Top The List Of The Most Costly Conditions In The United States: $201 Billion. Health Aff (Millwood) 2016; 35:1130-1135.
Bloom D E, Cafiero E T, Jané-Llopis E, Abrahams-Gessel S, Bloom L R, Fathima S, Feigl A B, Gaziano T, Mowafi M, Pandya A, Prettner K, Rosenberg L, Seligman B, Stein A, Weinstein, C. (2011). The Global Economic Burden of Non-communicable Diseases. Geneva: World Economic Forum.
Etkin A, Wager T D: Functional neuroimaging of anxiety: a meta-analysis of emotional processing in PTSD, social anxiety disorder, and specific phobia. Am J Psychiatry 2007; 164:1476-1488.
Etkin A, Patenaude B, Song Y J, Usherwood T, Rekshan W, Schatzberg A F, Rush A J, Williams L M: A cognitive-emotional biomarker for predicting remission with antidepressant medications: a report from the iSPOT-D trial. Neuropsychopharmacology 2015; 40:1332-1342.
Gyurak A, Patenaude B, Korgaonkar M S, Grieve S M, Williams L M, Etkin A: Frontoparietal Activation During Response Inhibition Predicts Remission to Antidepressants in Patients With Major Depression. Biol Psychiatry 2016; 79:274-281.
Liston C, Chen A C, Zebley B D, Drysdale A T, Gordon R, Leuchter B, Voss H U, Casey B J, Etkin A, Dubin M J: Default mode network mechanisms of transcranial magnetic stimulation in depression. Biol Psychiatry 2014; 76:517-526.
Chen A C, Oathes D J, Chang C, Bradley T, Zhou Z W, Williams L M, Glover G H, Deisseroth K, Etkin A: Causal interactions between fronto-parietal central executive and default-mode networks in humans. Proc Natl Acad Sci USA 2013; 110:19944-19949
Finn E S, Shen X, Scheinost D, Rosenberg M D, Huang J, Chun M M, Papademetris X, Constable R T: Functional connectome fingerprinting: identifying individuals using patterns of brain connectivity. Nat Neurosci 2015; 18:1664-1671.
Ferenczi E A, Zalocusky K A, Liston C, Grosenick L, Warden M R, Amatya D, Katovich K, Mehta H, Patenaude B, Ramakrishnan C, Kalanithi P, Etkin A, Knutson B, Glover G H, Deisseroth K: Prefrontal cortical regulation of brainwide circuit dynamics and reward-related behavior. Science 2016; 351:aac9698.
Etkin A, Egner T, Peraza D M, Kandel E R, Hirsch J: Resolving emotional conflict: a role for the rostral anterior cingulate cortex in modulating activity in the amygdala. Neuron 2006; 51:871-882.
Etkin A, Prater K E, Hoeft F, Menon V, Schatzberg A F: Failure of anterior cingulate activation and connectivity with the amygdala during implicit regulation of emotional processing in generalized anxiety disorder. Am J Psychiatry 2010; 167:545-554.
Etkin A, Schatzberg A F: Common abnormalities and disorder-specific compensation during implicit regulation of emotional processing in generalized anxiety and major depressive disorders. Am J Psychiatry 2011; 168:968-978.
Ernst M: Conflict adaptation in generalized anxiety disorder: small paradigm twist, large scientific leap. Am J Psychiatry 2010; 167:489-492.
Etkin A, Buchel C, Gross J J: The neural bases of emotion regulation. Nat Rev Neurosci 2015; 16:693-700.

### ADDITIONAL EMBODIMENTS

### Embodiment 1

A method of treating a brain dysfunction in a subject in need thereof, the method comprising: administering a first plurality of non-invasive stimulations to a first plurality of different brain regions of the subject; measuring a first plurality of responses to the first plurality of non-invasive stimulations; determining a dysfunctional brain network circuit in the subject based on the measuring of the first plurality of responses; administering a second plurality of non-invasive stimulations, based on the determining of the dysfunctional brain network circuit, to a second plurality of different brain regions of the subject, thereby treating the brain dysfunction in the subject.

### Embodiment 2

The method of embodiment 1, wherein said second plurality of non-invasive stimulations is a subset of said first plurality of non-invasive stimulations and said second plurality of different brain regions is a subset of said first plurality of different brain regions.

### Embodiment 3

The method of embodiment 1, wherein said determining a dysfunctional brain network circuit comprises comparing the measured responses in the subject to the responses measured in a healthy control.

### Embodiment 4

The method of embodiment 1 or 3, wherein said first plurality of different brain regions of the subject comprises two or more brain regions selected from the group consisting of frontal cortex, temporal cortex, parietal cortex, occipital cortex, hippocampus, amygdala, striatum or brainstem, and subregions thereof.

### Embodiment 5

The method of any of embodiments 1-4, wherein the non-invasive stimulation is transcranial magnetic stimulation (TMS), focused ultrasound stimulation, transcranial direct current stimulation or transcranial alternating current stimulation.

### Embodiment 6

The method of embodiment 5, wherein the TMS is selected from the group consisting of repetitive TMS (rTMS), single pulse TMS (spTMS) and paired pulse TMS (ppTMS).

### Embodiment 7

The method of any of embodiments 1-6, wherein the non-invasive stimulation is administered to a left dorsolateral prefrontal cortex (DLPFC), right DLPFC, dorsal cingulate, dorsomedial prefrontal cortex, frontopolar cortex, and/or ventrolateral prefrontal cortex of the subject.

### Embodiment 8

The method of any of embodiments 1-7, wherein the response is measured via electroencephalogram (EEG), Magnetoencephalography (MEG), Functional magnetic resonance imaging (fMRI), and/or Near-infrared spectroscopy (NIRS).

### Embodiment 9

The method of embodiment 8, wherein the response is measured via EEG or fMRI concurrently with or immediately after the TMS (TMS/EEG or TMS/fMRI).

### Embodiment 10

A method of treating a brain dysfunction in a subject in need thereof, the method comprising: administering a first plurality of non-invasive stimulations to a first plurality of different brain regions of the subject; measuring a first plurality of responses to the first plurality of non-invasive stimulations, thereby determining a brain region response for said subject; administering a second plurality of non-invasive stimulations, based on said brain region response, to a second plurality of different brain regions of the subject thereby treating said brain dysfunction, wherein said second plurality of non-invasive stimulations is a subset of said first plurality of non-invasive stimulations and said second plurality of different brain regions is a subset of said first plurality of different brain regions.

### Embodiment 11

A method of detecting a brain abnormality in a subject that has not been diagnosed with a clinical brain dysfunction or disorder, the method comprising: administering a non-invasive stimulation to a plurality of different brain regions of said subject, wherein said non-invasive stimulation to each said plurality of different brain regions is optionally different; measuring a response to said non-invasive stimulation in each of said plurality of different brain regions thereby obtaining a measured response in each of said plurality of different brain regions; and identifying the measured response as being an abnormal response by at least applying, to the measured response, a machine learning model, thereby detecting a brain abnormality.

### Embodiment 12

The method of embodiment 11, wherein said measuring a response is the measurement of a timing of the response, a magnitude in response, a frequency of the response, a duration of the response, and/or a presence or an absence of the response.

### Embodiment 13

The method of embodiment 11, wherein said response indicates an over activity of said brain regions or an under activity of said brain regions.

### Embodiment 14

The method of embodiment 13, further comprising treating said subject with repetitive transcranial magnetic stimulation (rTMS) where an over activity is measured.

### Embodiment 15

The method of embodiment 13, further comprising treating said subject with rTMS where an under activity is measured.

### Embodiment 16

The method of embodiment 11, wherein said each plurality of different brain regions of the subject is selected from the group consisting of frontal cortex, temporal cortex, parietal cortex, occipital cortex, hippocampus, amygdala, striatum or brainstem, and subregions thereof.

### Embodiment 17

The method of embodiment 11, wherein the non-invasive stimulation is administered to a left dorsolateral prefrontal cortex (DLPFC), right DLPFC, dorsal cingulate, dorsomedial prefrontal cortex, frontopolar cortex, and/or ventrolateral prefrontal cortex of the subject.

### Embodiment 18

The method of embodiment 11, wherein the non-invasive stimulation is TMS, focused ultrasound stimulation, transcranial direct current stimulation or transcranial alternating current stimulation.

### Embodiment 19

The method of embodiment 18, wherein the TMS is selected from the group consisting of rTMS, single pulse TMS (spTMS) and paired pulse TMS (ppTMS).

### Embodiment 20

The method of embodiment 11, wherein the response is measured via electroencephalogram (EEG), Magnetoencephalography (MEG), Functional magnetic resonance imaging (fMRI), and/or Near-infrared spectroscopy (NIRS).

### Embodiment 21

The method of embodiment 20, wherein the response is measured via EEG or fMRI concurrently with or immediately after the TMS (TMS/EEG or TMS/fMRI).

### Embodiment 22

The method of embodiment 11, wherein the machine learning model comprises a neural network, a regression model, an instance-based model, a regularization model, a decision tree, a Bayesian model, a clustering model, an associative model, a deep learning model, a dimensionality reduction model, and/or an ensemble model.

### Embodiment 23

The method of embodiment 11, wherein the machine learning model is trained to identify one or more abnormal responses.

### Embodiment 24

The method of embodiment 23, further comprising: training, based at least on training data, the machine learning model, the training data including a plurality of abnormal responses and/or normal responses, and the machine learning model being trained to differentiate between the abnormal responses and the normal responses.

### Embodiment 25

The method of embodiment 24, wherein the training data includes a plurality of variables associated with abnormal responses and/or normal responses, and wherein the plurality of variables includes a timing of the response, a magnitude in response, a frequency of the response, a duration of the response, and/or a presence or an absence of the response.

### Embodiment 26

A method of detecting a brain abnormality in a subject that has not been diagnosed with a clinical brain dysfunction or disorder, the method comprising: administering a non-invasive stimulation to a plurality of different brain regions of said subject, wherein said non-invasive stimulation to each said plurality of different brain regions is optionally different; measuring a response to said non-invasive stimulation in each of said plurality of different brain regions thereby obtaining a measured response in each of said plurality of different brain regions; and comparing the measured response to a control response thereby detecting a brain abnormality in the subject.

### Embodiment 27

The method of embodiment 26, wherein said measuring a response is the measurement of a timing of the response, a magnitude in response, a frequency of the response, a duration of the response, and/or a presence or an absence of the response.

### Embodiment 28

The method of embodiment 26, wherein said response indicates an over activity of said brain regions or an under activity of said brain regions.

### Embodiment 29

The method of embodiment 28, further comprising treating said subject with repetitive transcranial magnetic stimulation (rTMS) where an over activity is measured.

### Embodiment 30

The method of embodiment 28, further comprising treating said subject with rTMS where an under activity is measured.

### Embodiment 31

The method of embodiment 26, wherein said each plurality of different brain regions of the subject is selected from the group consisting of frontal cortex, temporal cortex, parietal cortex, occipital cortex, hippocampus, amygdala, striatum or brainstem, and subregions thereof.

### Embodiment 32

The method of embodiment 26, wherein the non-invasive stimulation is administered to a left dorsolateral prefrontal cortex (DLPFC), right DLPFC, dorsal cingulate, dorsomedial prefrontal cortex, frontopolar cortex, and/or ventrolateral prefrontal cortex of the subject.

### Embodiment 33

The method of embodiment 26, wherein the non-invasive stimulation is TMS, focused ultrasound stimulation, transcranial direct current stimulation or transcranial alternating current stimulation.

### Embodiment 34

The method of embodiment 33, wherein the TMS is selected from the group consisting of rTMS, single pulse TMS (spTMS) and paired pulse TMS (ppTMS).

### Embodiment 35

The method of embodiment 26, wherein the response is measured via electroencephalogram (EEG), Magnetoencephalography (MEG), Functional magnetic resonance imaging (fMRI), and/or Near-infrared spectroscopy (NIRS).

### Embodiment 36

The method of embodiment 35, wherein the response is measured via EEG or fMRI concurrently with or immediately after the TMS (TMS/EEG or TMS/fMRI).

What is claimed is:

1. A method of treating a brain dysfunction in a subject in need thereof, the method comprising:
administering a first plurality of non-invasive stimulations to a first plurality of different brain regions of the subject;
measuring a first plurality of responses to said first plurality of non-invasive stimulations;
determining a dysfunctional brain network circuit in the subject based on said measuring of said first plurality of responses;
administering a second plurality of non-invasive stimulations, based on the determining of said dysfunctional brain network circuit, to a second plurality of different brain regions of the subject, thereby treating the brain dysfunction in the subject.

2. The method of claim 1, wherein said second plurality of non-invasive stimulations is a subset of said first plurality of non-invasive stimulations and said second plurality of different brain regions is a subset of said first plurality of different brain regions.

3. The method of claim 1, wherein said determining a dysfunctional brain network circuit comprises comparing the measured responses in the subject to the responses measured in a healthy control.

4. The method of claim 1, wherein said first plurality of different brain regions of the subject comprises two or more brain regions selected from the group consisting of frontal cortex, temporal cortex, parietal cortex, occipital cortex, hippocampus, amygdala, striatum or brainstem, and subregions thereof.

5. The method of claim 1, wherein the non-invasive stimulation is transcranial magnetic stimulation (TMS), focused ultrasound stimulation, transcranial direct current or transcranial alternating current stimulation.

6. The method of claim 5, wherein the TMS is selected from the group consisting of repetitive TMS (rTMS), single pulse TMS (spTMS) and paired pulse TMS (ppTMS).

7. The method of claim 1, wherein the non-invasive stimulation is administered to a left dorsolateral prefrontal cortex (DLPFC), right DLPFC, dorsal cingulate, dorsomedial prefrontal cortex, frontopolar cortex, and/or ventrolateral prefrontal cortex of the subject.

8. The method of claim 1, wherein the response is measured via electroencephalogram (EEG), Magnetoencephalography (MEG), Functional magnetic resonance imaging (fMRI), and/or Near-infrared spectroscopy (NIRS).

9. The method of claim 8, wherein the response is measured via EEG or fMRI concurrently with or immediately after the TMS (TMS/EEG or TMS/fMRI).

10. A method of treating a brain dysfunction in a subject in need thereof, the method comprising:
administering a first plurality of non-invasive stimulations to a first plurality of different brain regions of the subject;
measuring a first plurality of responses to said first plurality of non-invasive stimulations, thereby determining a brain region response for said subject;
administering a second plurality of non-invasive stimulations, based on said brain region response to a second plurality of different brain regions of the subject thereby treating said brain dysfunction,
wherein said second plurality of non-invasive stimulations is a subset of said first plurality of non-invasive stimulations and said second plurality of different brain regions is a subset of said first plurality of different brain regions.

11. A method of detecting a brain abnormality in a subject that has not been diagnosed with a clinical brain dysfunction or disorder, the method comprising:
administering a non-invasive stimulation to a plurality of different brain regions of said subject, wherein said non-invasive stimulation to each of said plurality of different brain regions is optionally different;
measuring a response to said non-invasive stimulation in each of said plurality of different brain regions thereby obtaining a measured response in each of said plurality of different brain regions; and
identifying the measured response as being an abnormal response by at least applying, to the measured response, a machine learning model, thereby detecting a brain abnormality.

12. A method of detecting a brain abnormality in a subject that has not been diagnosed with a clinical brain dysfunction or disorder, the method comprising:
administering a non-invasive stimulation to a plurality of different brain regions of said subject, wherein said non-invasive stimulation to each of said plurality of different brain regions is optionally different;
measuring a response to said non-invasive stimulation in each of said plurality of different brain regions thereby obtaining a measured response in each of said plurality of different brain regions;
comparing the measured response to a control response thereby detecting a brain abnormality in the subject.
